(19) United States
(12) Patent Application Publication
(10) Pub. No.: US 2023/0272372 A1
(43) Pub. Date: Aug. 31, 2023

(54) Cellular Coding Constructs Providing Identification of Cellular Entities

(71) Applicants: LASE Innovation Inc., Woburn, MA (US); The General Hospital Corporation, Boston, MA (US)

(72) Inventors: Seok Hyun Yun, Belmont, MA (US); Yue Wu, Cambridge, MA (US); Nicola Martino, Cambridge, MA (US); Sheldon J J. Kwok, Boston, MA (US)

(21) Appl. No.: 17/889,811

(22) Filed: Aug. 17, 2022

Related U.S. Application Data
(60) Provisional application No. 63/234,076, filed on Aug. 17, 2021.

(51) Int. Cl. C12N 15/10; C12Q 1/6881

(52) U.S. Cl. CPC C12N 15/1065; C12Q 1/6881

(57) ABSTRACT

A cellular coding construct uniquely codes a cellular entity and includes a laser particle and a structurally coded oligonucleotide. The structurally coded oligonucleotide and the laser particle have a physical association with each other and are configured for physical association with the cellular entity and also configured for distinctive identification of the cellular entity.

### CROSS-REFERENCE TO RELATED APPLICATIONS

The present application claims priority to U.S. Provisional Pat. Application Serial No. 63/234,076, entitled “Cellular Coding Constructs Providing Identification of Cellular Entities” and filed Aug. 17, 2021. The foregoing application is incorporated herein by reference in its entirety.

### SEQUENCE LISTING

The instant application contains a Sequence Listing which has been submitted electronically in XML format and is hereby incorporated by reference in its entirety. Said XML copy, created on Mar. 22, 2023, is named 4657_1007_SL.xml and is 123,716 bytes in size.

### TECHNICAL FIELD

The present invention relates to identification of cellular entities for purposes of analysis and more particularly to such identification using both microparticles providing laser emission and oligonucleotide sequences in physical association with such cellular entities.

### BACKGROUND ART

Cells are the fundamental building blocks of all life forms. Understanding cells from their shapes to molecular content, gene expression, functions, and to trajectories, as well as interactions with other cells and surrounding environment is a cornerstone of life sciences. There have been significant advances in cell analysis. Single-cell sequencing led the paradigm shift in analyzing cells from ensembles to individual cells. This breakout success motivated the development of various new techniques that couple imaging to sequencing and antibodies to sequencing for multi-dimensional analysis at the molecular, cellular, and tissue levels.

Cells are dynamic entities, changing over time and responsive to their environment. Unfortunately, the current single-cell sequencing techniques, including droplet-based sequencing and in situ sequencing, are exclusively performed at the terminal stage of analysis ex vivo, and thus cannot easily probe dynamic cellular processes. These techniques require direct readout of target RNAs and DNAs that are either in the cytoplasm of fixed cells or released after lysing cells.

On the other hand, optical microscopy can visualize live cells repeatedly and can be used to measure the temporal changes, spatial movement, and behaviors of the cells. Conventional fluorescent dyes, proteins, and nanoparticles provide limited optical channel (<100) to track individual cells or groups of cells and to obtain their dynamic information in situ.

A new technology based on laser-emitting particles can provide spectral features that can serve as optical barcodes of cells and promise to enable large-scale (>1,000) optical tracking and imaging of thousands to millions of cells. However, given the constraints imposed by live cells, such as limited fluorescent channels available for multiplexing and the need to minimize perturbations on cells, it is difficult to obtain comprehensive molecular information using optical microscopy. Optical imaging techniques, including transgenic reporter proteins and in situ hybridization, have thus far allowed only a relatively limited number of genes and proteins to be analyzed, whereas ex vivo single-cell sequencing techniques can analyze a greater number of genes and proteins, are much faster and available in most of the single-cell analysis cores.

### SUMMARY OF THE EMBODIMENTS

In accordance with one embodiment of the invention, there is provided a cellular coding construct that uniquely codes a cellular entity. In this embodiment, the cellular coding construct includes: a laser particle; and a structurally coded oligonucleotide, wherein the structurally coded oligonucleotide and the laser particle have a physical association with each other and are configured for physical association with the cellular entity and also configured for distinctive identification of the cellular entity.

In a related embodiment, the invention further includes a non-volatile storage arrangement encoded with identification data characterizing the structurally coded oligonucleotide and the laser particle and their physical association.

Optionally, the laser particle and the structurally coded oligonucleotide have a combined dimension that is less than 3 µm. Optionally, the cellular construct is physically associated with a specified cellular entity. Also optionally the non-volatile storage arrangement is further encoded with biological data characterizing the specified cellular entity. As a further option, the biological data is genetic sequence data. As a further option, the cellular construct further includes a linker configured to physically attach the cellular construct to the cellular entity.

In a related embodiment, there is provided a population of objects wherein each object is a distinct cellular construct in accordance with any of the previous descriptions. In a related embodiment, the structurally coded oligonucleotide includes a plurality of ligated sequence segments. Optionally, the physical association between the structurally coded oligonucleotide and the laser particle may be configured for disassociation.

In another embodiment of the invention, there is provided a non-volatile storage arrangement encoded with data characterizing a structurally coded oligonucleotide and a laser particle physically associated with the structurally coded oligonucleotide, and for each identifier is provided, pertinent to the corresponding cellular entity, information selected from the group consisting of DNA data, RNA data, protein data, morphology data, location data, functional data, and behavioral data. Optionally, the structurally coded oligonucleotide and the laser particle are physically associated with the cellular entity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIG. 1A shows a schematic of a typical conventional oligo-barcoded microbead and its utility in single-cell analysis of RNA expression. FIG. 1B shows another prior-art example of the utility of the conventional oligo-barcoded microbead in single-cell analysis of cell-surface protein expression. The oligo-barcoded microbead contains one or multiple types of capture sequences that are complementary to specific oligo-sequences in the RNA and feature-barcoded antibodies.

FIGS. 2A through 2C show different oligonucleotide-coated, optical microparticles known in the prior art. FIG. 2A shows an oligo-coated fluorescent microsphere used for multiplexed assay. FIG. 2B shows an oligo-barcoded microsphere embedding optically barcoding elements. FIG. 2C illustrates conventional microdisk laser particles.

FIGS. 3A through 3C show a multi-barcoding laser particle, which contains multiple lasing disks providing an optical barcode and oligonucleotide sequences providing a molecular barcode, in accordance with an embodiment of the present invention. FIG. 3A depicts the structure of a barcoding construct consisting of a triplet laser particle and an oligo barcode. FIG. 3B depicts a process of attaching oligo sequences containing two oligo barcoding segments to a laser particle. FIG. 3C depicts another process of attaching oligo sequencies using ligation.

FIGS. 4A through 4D show a schematic of multi-barcoded microparticles and their utilities for tagging cells, in accordance with another embodiment of the present invention. FIG. 4A shows a microparticle capable of producing an optical barcoding feature and oligonucleotide barcoding sequence. FIG. 4B shows such a microparticle in the cytoplasm. FIG. 4C shows such a microparticle attached to the cell membrane. FIG. 4D shows such a microparticle attached to the nuclear membrane of a cell.

FIGS. 5A and 5C illustrate a method for capturing mRNA and oligo barcodes released from a multi-barcoded microparticle by primer sequences, producing cDNA for single cell sequencing, in accordance with embodiments of the present invention. FIG. 5A illustrates the use of a microfluidic device configured to capture the oligo barcode sequences from optical microparticles in cells. FIGS. 5B and 5C illustrate a process wherein the oligo barcode is released from a multi-barcoded microparticle. In FIG. 5B, the primers are not released from a sequencing bead (e.g., Drop-seq), whereas in FIG. 5C, the primers can be released from a sequencing bead (e.g., 10x Genomics and inDrop).

FIG. 6 illustrates a split-pool method to attach different oligo barcodes to different LPs in a large scale. An example of oligo barcode sequence attached to an LP is also shown.

FIGS. 7A through 7E are experimental data that indicate the presence of oligo barcodes on three different microparticles. FIG. 7B shows a PCR result and fluorescence in situ hybridization images of oligo-coated semiconductor-disk laser particles. FIG. 7B shows a scanning electron microscopy image of oligo-coated triplet laser particles and PCR gel electrophoresis data of the dual-barcoding triplet laser particles. FIG. 7C shows a PCR gel electrophoresis data and fluorescence in situ hybridization image of a triplet laser particle coated with oligo barcodes in three stages. FIG. 7D shows another results obtained by using triplet laser particles fabricated with reverse transcription method. FIG. 7E shows results obtained by using triplet laser particles fabricated with the ligation method.

FIGS. 8A and 8B show experimental data obtained with dual-barcoding laser particles produced by using a split-pool method. FIG. 8A shows images of cells prior to sequencing and electrophoresis data. FIG. 8B shows results of single-cell sequencing.

FIGS. 9A and 9B show a non-volatile storage arrangement encoded with the identification data of dual-barcoding microparticles. FIG. 9A shows an exemplary identification data in the non-volatile storage arrangement for a single barcoding construct. FIG. 9A discloses SEQ ID NOS 30-31, respectively, in order of appearance. FIG. 9B shows an exemplary set of identification data in the non-volatile storage arrangement for a population of distinct barcoding constructs. Column “Oligo 1” in FIG. 9B discloses SEQ ID NOS 30 and 32-34, respectively, in order of appearance. Column “Oligo 2” in FIG. 9B discloses SEQ ID NOS 31, 35, 31, and 36, respectively in order of appearance.

FIGS. 10A through 10C show three flow charts showing the utilities of barcoded microparticles for single cell analysis.

FIG. 11 show four different types of cell samples, namely cells injected into animals, cells in 3-dimensional culture, cells in blood, and cells in well plates.

FIG. 12 provides exemplary workflows using the barcoded LPs to track cells across different measurement technologies and instruments, such as microscopy, flow cytometry, and sequencing. Barcoded cells may be pooled between measurements.

FIG. 13 shows an embodiment of a non-volatile storage arrangement encoded with biological data characterizing a sample of cells along with the identifiers of dual-barcoding microparticles pertinent to the corresponding cells.

FIG. 14 provides a simplified diagram showing various data processing steps to analyze the biological data, such as DNA data, RNA data, protein data, morphology data, location data, functional data, and behavioral data, that are obtained by using cellular barcoding constructs.

FIG. 15 provide two different methods to tag cells in tissues with barcoded microparticles, one using a patterned array of microparticles and the other using free falling or ballistically projected microparticles.

FIG. 16 illustrates a microfluidic arrangement for capturing single nuclei tagged with barcoded LPs for ATAC sequencing.

FIG. 17 illustrates a method for tagging multiple groups of cells with barcoded LPs. LPs used in each cell group has a common oligo sequence in their oligo barcodes to facilitate analysis.

FIG. 18 illustrates a modified process for sci-Seq or SPLiT-seq where the cells are tracked based on their optical barcodes through different well plates during a split and pool oligo barcoding step.

### DETAILED DESCRIPTION OF SPECIFIC EMBODIMENTS

Definitions. As used in this description and the accompanying claims, the following terms shall have the meanings indicated, unless the context otherwise requires:

A “cellular entity” includes a cell, or a part of a cell, such as a nucleus, vesicle or organelle, or a coherent organization of cells, such as tissue and multicellular spheroid. The cellular entity may be live or chemically fixed.

A “sample” refers to a group of cellular entities that are to be or have been analyzed, which are typically prepared and carried in a single container, well plate, or vial.

A “microparticle” is a three-dimensional particle with a size smaller than 100 µm. A particle having a size of 10 nm is still a “microparticle” in this context, because it has a size smaller than 100 microns.

An “optical barcode” is an optically distinguishable feature, such as shape, color, or particular emission spectrum, which can be read optically and associated with a cellular entity to serve as an identification of the cellular entity.

An “oligo barcode” or “molecular barcode” is an oligonucleotide sequence that can be uniquely assigned to a single cellular entity or a sample. The words oligonucleotide sequence or oligo barcode is typically referred to a specific series of nucleotide codes; however, they are often used to refer to the actual molecule that contains the series of nucleotides.

An “optical microparticle” is a microparticle providing an optical barcode without molecular barcode.

A “dual-barcoding” or “multi-barcoding” particle is a microparticle capable of providing both optical and oligo barcodes. The optical and oligo barcodes constitute the identification data associated with the particle.

A “laser particle” or an “LP” is a microparticle capable of emitting coherent light when inquired by a suitable excitation. The output spectrum preferably consists of discrete narrowband laser lines, which are typically related to the particular geometry and composition thereof, which serve as an optical barcode of the cellular entity associated with the laser particle. An LP without oligonucleotides is an optical microparticle. An LP with a molecular barcode is a multi-barcoding particle.

To “tag” a cellular entity means to cause one or more barcoding particles to be physically associated with the cellular entity. For cells, tagging is achieved by attaching the barcoding particle(s) on the cell membrane or inserting the particle(s) into the cytoplasm.

To “track” a cellular entity means to identify the tagged cellular entity based on its barcoding microparticle(s) over time, in space, across instruments, processes, or analyses.

A “physical association” between an oligonucleotide and a laser particle and between a cellular construct and cellular entity is established in each instance by a structural agent selected from the group consisting of direct chemical bonding, a linker, encapsulation, and any other form of physical confinement. Two physically associated items are in proximity with each other typically, although not necessarily, within 100 nm or in some cases within 10 nm or less.

A “physical disassociation” of an oligonucleotide from a laser particle occurs if a physical association between them has been disrupted. Such disruption can be achieved by breaking the structural agent causing the physical association, such breaking by a method selected from the group consisting of breaking a direct chemical bond, breaking a linker, and breaking a physical encapsulation or other form of physical confinement.

To make a “distinctive identification” of a cellular entity includes an activity selected from the group consisting of (a) making a unique identification of the cellular entity and (b) identifying cellular entities having a specified set of attributes in common. When the cellular entity is tagged with more than one cellular construct each with different identification data, the distinctive identification of the cellular entity is determined by one of, a fraction, or all of the identification data of the cellular constructs associated with the cellular entity.

It is expected that our ability to acquire multi-dimensional single-cell information could be greatly enhanced if individual cells can be tagged with barcoding features that are compatible with both optical imaging, which is noninvasive and thus suited for obtaining dynamic information, and single-cell sequencing, which is invasive but suited for obtaining comprehensive molecular information. The optical barcodes of cells can be read optically in real time and repeatedly as needed, and the oligo barcodes of cells can be read using sequencing. These cells can be imaged in vivo, then analyzed in flow, and then sequenced, for example. Recording the optical barcode in situ makes it possible to compile all the data from the same cell acquired at different times, locations, and apparatuses. The acquired data can then be all aligned to individual cells according to their unique barcoding features and integrated to reveal the biology of the cells.

The synergistic combination of large-scale optical barcodes and oligonucleotide barcodes can offer many different new ways to analyze cells comprehensively. This innovation may change the way we use multi-dimensional single-cell analysis for scientific discovery and diagnostic and therapeutic applications in healthcare.

Technologies are available to label a large number of cells, typically from 100 to 100,000 cells, with uniquely varying oligonucleotide sequences, called oligo barcodes or DNA barcodes. These molecular barcodes, which are typically read by using next-generation sequencing technologies or fluorescence in-situ hybridization (FISH), have been the key enabler in droplet-based single-cell transcriptomics and proteomics analysis and spatial transcriptomics based on patterned barcodes on slides.

Manufacture and characteristics of laser microparticles are described in published PCT Application WO2017/210675, which is hereby incorporated herein by reference. The present application describes a new use and context for associating microparticles with sequencing information of samples.

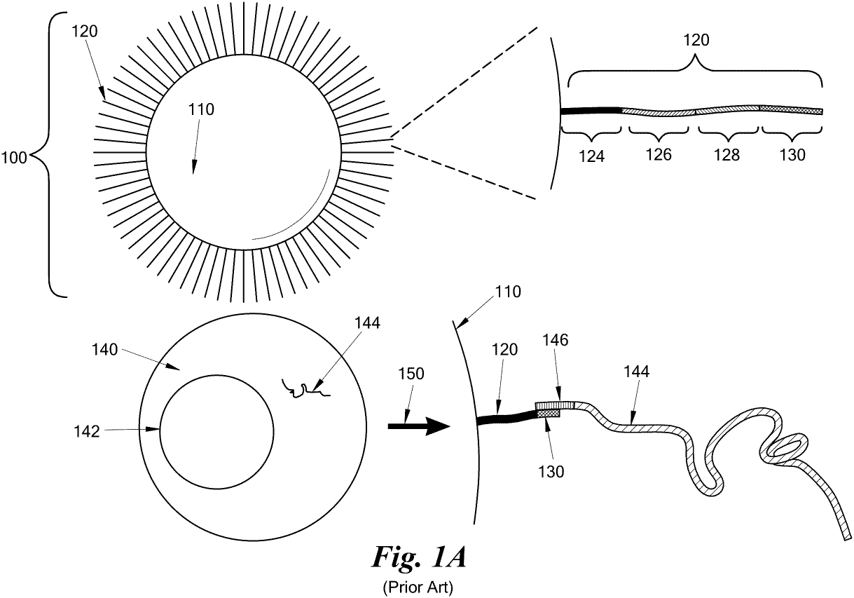

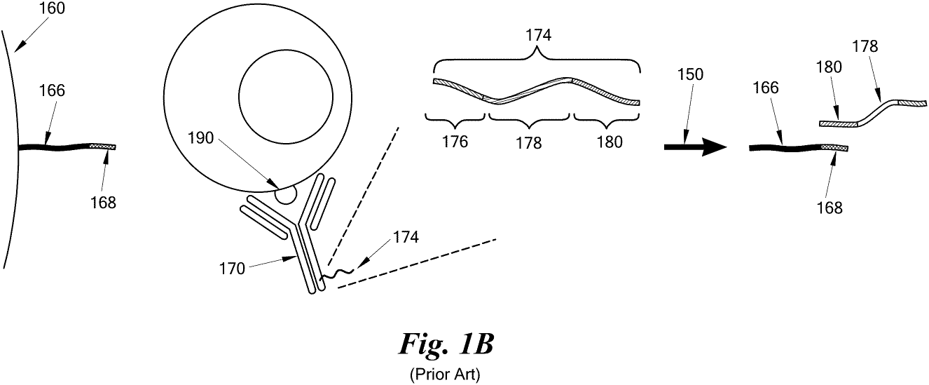

FIG. 1A depicts a conventional cellular barcoding scheme that is widely used for single-cell sequencing³⁻⁵. It is based on a barcoding microbead 100 that has a microbead 110 and surface-coated, oligonucleotide sequences 120. The oligo sequence has multiple segments including a primer site 124, cell barcode 126, unique molecular index (UMI) 128, and capture sequence 130. The typical length of the primer site is 22 nucleotides (nt), that of the barcode is 16 nt, and that of UMI is 10 nt. Different types of barcoded microbeads have been demonstrated. The microbeads are made of solid polymer spheres or soft gels. One of the widely used designs is the gel bead-in emulsion (GEM) technology developed by 10x Genomics. The gel microbeads used in the GEM technology have a typical diameter of 70-85 µm. The Drop-seq technology used polystyrene beads with diameters of 10-30 µm.

The cell barcode 126 provides the unique tag to a sample that is specifically associated with the microbead 100. On a single microbead 110, a large number, over 1 million copies, of oligo sequences 120 are conjugated, each of which has the same cell barcode 126 but different UMI 128. The capture sequence 130 is one of several different types, such as (i) oligo deoxythymine (dT) (termed poly(dT)), (ii) a complementary sequence to specific “feature barcodes”, or (iii) template switch oligo (TSO).

The poly(dT) capture sequence is used to capture RNA molecules released from cells. Consider a cell 140 with a nucleus 142 and intracellular RNA 144. When the cell is lysed (process 150) in proximity of the microbead 100, the cellular content released from the cell comes into in contact with the oligo sequences 120, and the common poly(dA) tail 146 of the released RNA 144 is hybridized with the poly(dT) capture sequence 130. The oligo segment 120 typically has a linear structure without hairpin portions. Intracellular RNA 144 may include hairpin portions.

FIG. 1B illustrates the feature barcode technology. This method adds extra channels of information to cells by running single-cell gene expression in parallel with other assays. This technology is used for measuring cell surface protein expression levels via antibody or antigen-multimer staining assays. Also, feature barcoding can be used for multiplexing sample populations using antibody-based hashtag oligos (HTOs) or CRISPR screening. To utilize feature barcoding, a microbead 160 is coated with oligo sequences 166 containing a capture sequence 168, as well as the RNA capture sequences 120. An antibody 170 is conjugated with an oligo sequence 174, which typically includes a PCR primer 176, antibody-specific feature barcode 178, and complementary capture sequence 180. The complementary capture sequence 180 may be poly(dA). In this case, the capture sequence 168 is poly(dT). More generally, the capture sequence 180 may be a specific oligonucleotide, such as TotalSeq™ B or TotalSeq™ C, and the capture sequence 168 is its complementary sequence. The antibody 170 is bound to its target molecule 190 on the cell surface. The cell is lysed near the bead 160, and the capture sequence 168 is hybridized with its complement 180. The measurement of the number of the feature barcodes 178 allows the user to determine the expression level of the cell-surface marker 190 of the cell. This technique is known as CITE-seq⁶ or REAP-seq⁷. The 10x Genomics GEM technology offers sequencing microbeads containing multiple capture sequences, such as both poly(dT) and TotalSeq™ B.

The feature barcoding also allows for the analysis of gene expression changes caused by the presence of CRISPR perturbations in Perturb-seq type assays. Cells are transduced with a pooled lentiviral library containing guide RNAs (gRNAs) targeting many genes in a genome. These libraries can be designed for common CRISPR applications including genetic knockout, activation, cutting, and repression. The Feature barcode technology is used to assess the effects of perturbations on gene expression via direct capture of gRNAs and polyadenylated mRNAs from the same cell. This measurement is useful for analyzing regulatory gene networks and pathways involved in development and disease for resolving complex biological pathways and dissecting cellular regulation.

The capture sequence on the barcoded bead may be TSO, an oligo that hybridizes to untemplated C nucleotides added by the reverse transcriptase during reverse transcription (RT). The TSO adds a common 5′ sequence to full length cDNA that is used for downstream cDNA amplification. Compared to this single cell 5′ assay, the TSO is used differently in the single cell 3′ assay. In the 3′ assay, the poly(dT) or a capture sequence is part of the gel bead oligo, with the TSO supplied in the RT Primer. In the 5′ assay, the poly(dT) is supplied in the RT Primer, and the TSO is part of the gel bead oligo.

In the prior art examples depicted in FIG. 1, the microbeads 110 and 160 are typically optically inactive, or non-luminescent upon optical excitation. On the other hands, various types of photoluminescent particles, such as dye-doped microspheres and semiconductor nanocrystals, have been coupled with oligonucleotides for capturing specific complementary oligo sequences.

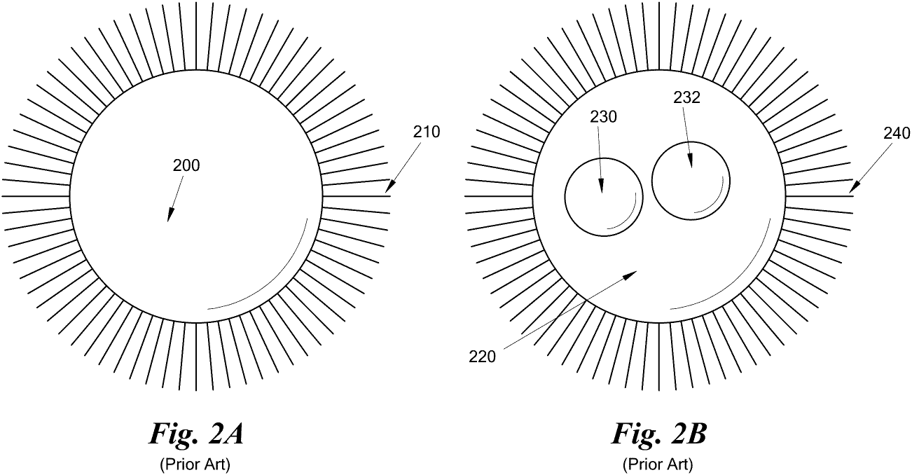

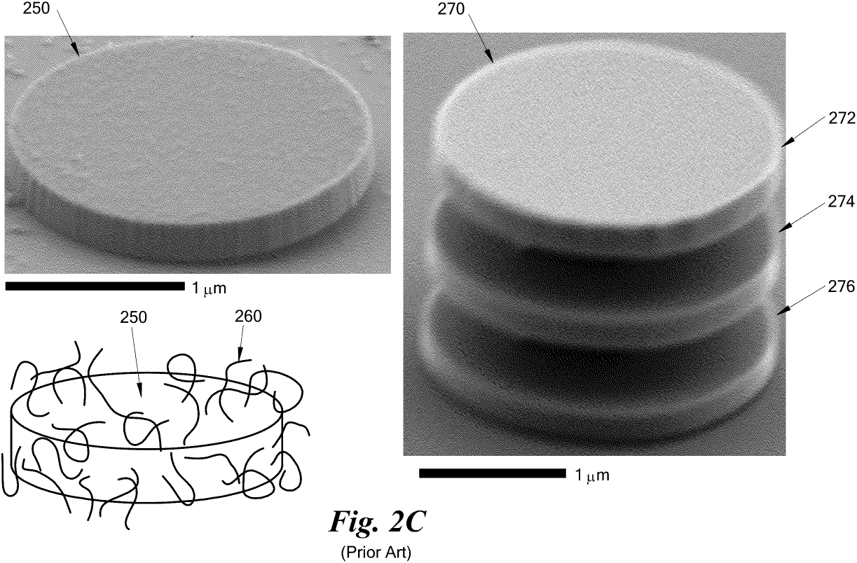

FIG. 2 depicts a few examples of luminescent particles known in the prior art. FIG. 2A shows an oligo-coupled polystyrene microbead 200, such as MagPlex-TAG™ microspheres commercialized by Luminex. These beads are dyed into spectrally distinct sets allowing them to be individually identified by fluorescence imaging or flow cytometry. The number of spectrally distinguished sets is typically less than 100, although multiplexing up to 500 has been claimed. Each of the color-coded beads has a unique 24 base DNA sequence, called an “anti-TAG,” covalently coupled to its surface. These beads enable the user to design custom bead arrays simply by adding a complementary “TAG” sequence to primers or probes of interest and hybridizing those primers or probes to the anti-TAG sequences on the addressable microsphere. The typical size of MagPlex-TAG™ microparticles is 5-7 µm.

FIG. 2B shows a microbead 220 that embeds a few optical particles, 230 and 232, and is coupled with oligonucleotide sequences 240 on the surface. The optical particles are configured to produce distinct optical emission spectra, which collectively serve as an optical barcode of the microbead 220. PCT/US2019/057320 describes such microparticles, in which the oligo sequence 240 is essentially the same as the RNA capture sequence 120 in the conventional oligo-barcoded microsphere 100 used for single-cell transcriptomics. The typical intended size of the combined microbead 220 is 10 to 60 µm.

FIG. 2C illustrates microdisk lasers, also known as laser particles (LPs). LPs are micron-sized biocompatible particles, each emitting coherent light with a unique spectrum, which serves as an optical barcode¹. PCT Application WO2017210675 describes a microdisk 250 capable of emitting sub-nanometer linewidth and teaches that such particles may be coated with polymers 260. More recently, U.S. Provisional Application No. 63/075,468 extended the embodiment to multiplet LPs. For example, a triplet LP 270 has three disks, 272, 274, and 276, that are physically associated, and each disk is configured to generate narrowband laser emission when sufficient optical pump energy is provided. The emission spectra of the three disks collectively constitute the optical barcode of the triple LP. However, combining LPs with oligo barcodes to generate unique identification data have not been described in the prior art.

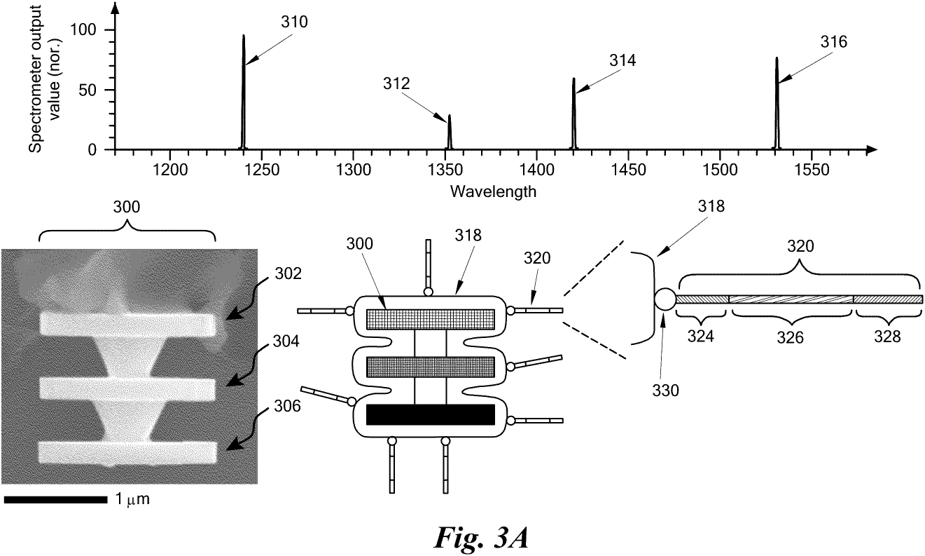

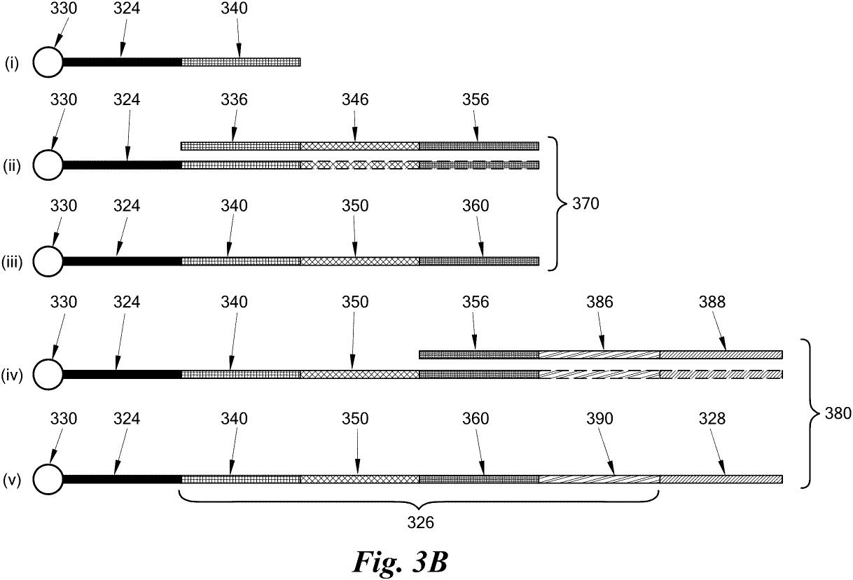

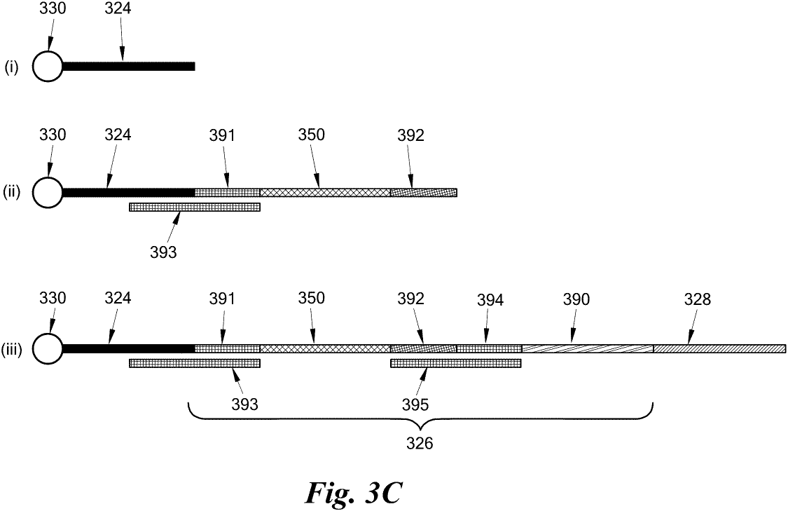

FIGS. 3A and 3B depict one embodiment of the present invention based on triplet LPs. A method to produce triplet LPs using semiconductors is described in US Provisional Application No. 63/075,468. FIG. 3A shows a scanning electron microscopy image of a typical triplet LP 300. When each disk generates a single spectral peak, the optical barcode of the LP has three lasing wavelengths. A single disk, however, may generate two spectral peaks corresponding to two different lasing modes. In this case, such a triplet LP can generate a total of 4 to 6 spectral peaks. An example in FIG. 3A illustrates four lasing peaks, 310 to 316.

The total number of possible optical barcodes obtainable from a set of disks is a function of the number of disks and the the number of possible wavelengths that can be ascribed to each disk. For a given semiconductor material composition, the number of distinguishable wavelengths is typically ~100 assuming a wavelength bin size of 1 nm over a spectral range of 100 nm. Assuming each triplet LP generates three independent lasing peaks, the total number of unique optical barcodes ranges from approximately ₁₀₀C₃ = 161,770 to 100³ = 1,000,000 depending on the overlap in the tuning ranges of the lasing peaks. Therefore, a population of triple LPs, each with random laser peaks, is suited for large-scale optical barcoding applications². For quartet LPs consisting of four independent microdisk lasers that are randomly sized, the number of optical barcodes is increased to, approximately, ₁₀₀C₄ = 3,921,225 up to ~ 100⁴ = 100 million depending on the overlap in the tuning ranges of the lasing peaks.

In FIG. 3A, the microparticle 300 is coated with protective material 318. An exemplary protective material is silica (SiO₂), but other materials such as polystyrene are possible. Several methods are available to conjugate oligonucleotides on the protective material. As an example, carboxyl (—COOH) group is introduced on the surface of the silica layer 318. 5′ amino-modified oligonucleotide 320 is then crosslinked to the carboxyl group via carbodiimide crosslinker chemistry by N-ethyl-N′-(3-dimethylaminopropyl)carbodiimide (EDC). Alternatively, the silica surface may be functionalized with an amine (—NH₂) group. An amine-reactive linker containing N-hydroxysuccinimidyl (NHS) ester at both ends, dithiobis(succinimidyl propionate (DTSP), is added to convert the surface of LPs to be NHS ester. Then, the 5′ end amino-modified oligonucleotides is conjugated to the surface. With DTSP, we may choose to insert a disulfide bond may be inserted to facilitate the oligo cleavage by reducing agents such as Tris(2-carboxyethyl) phosphine hydrochloride, or possibly the reducing agents in the 10x Chromium. Alternatively, the silica surface may be functionalized with a biotin group and streptavidin linker, and then the 3′ end of oligonucleotides can be attached by another biotin group.

The oligonucleotide sequence 320 can be identical, or similar, to that used in the feature barcoding technology described in FIG. 1C. For example, it is comprised of a PCR primer 324, a LP-specific oligonucleotide barcode 326, and a complementary capture sequence 328, as well as a linker 330. The complementary capture sequence 328 may be poly(dT) or a feature barcode capture sequence, such as the one in TotalSeq™ B. Optionally, the linker 330 may include a photocleavable, chemically cleavable, enzymatic cleavable, or chemically displaceable site, so that the oligo sequence is releasable or dissociable from the surface 318 of the LP upon ultraviolet (UV) irradiation or injection of a cleaving chemical, enzyme, or competitive analog. Preferably but not necessarily, all the oligonucleotides attached to a single LP have the identical sequence. The oligonucleotide 326 constitutes the oligo or molecular barcode of the LP.

Ideally, the majority, if not all, of the LPs in a population used in the analysis of a sample have different oligo barcodes and different optical barcodes. Then, the optical emission allows each LP to be distinguished from the others in the population. Likewise, the oligo barcode allows each particle to be distinguished from the others in the population of LPs. As described later in detail, the physical association between the optical and oligo barcodes can established in a number of ways. For example, the association can be formed during conjugation of the oligonucleotide sequences onto the LPs. In this case, once the optical barcode of an LP is measured, the oligo barcode on the LP is determined automatically.

The oligonucleotide barcode 326 may be a single oligo sequence or consist of more than one sequence concatenated in multiple stages. Several methods can be used to introduce an oligonucleotide sequence to the PCR handle, such as reverse transcription (RT, FIG. 3B) and DNA ligation (FIG. 3C). FIG. 3B shows a schematic of a two-stage oligo barcodes and five representative steps using RT to fabricate the oligo sequence. The fabrication process is similar to that used for fabricating conventional gel beads¹¹, which uses ligation and primer extension in combination with a split-and-pool manner. The steps are as follows: (i) After functionalizing the surface of an LP with the carboxyl group, an oligonucleotide sequence comprising the linker 330, the PCR adapter 324, and a first ligation site 340 is attached onto the functionalized surface of an LP using the EDC chemistry. (ii) Then, a first extension sequence, which includes a sequence 336 complementary to the first ligation site 340, a first complementary barcode sequence 346, and a second complementary ligation site 356, is hybridized to the first ligation site 340. Then, a primer extension reaction is performed to extend the sequence. The enzymatic extension may be performed at a relatively high temperature, such as 60° C., to minimize unspecific annealing of DNA primers and a thermostable DNA polymerase, such as Bst 2.0 DNA polymerase. This process inserts a first barcode sequence 350 and a second ligation site 360. (iii) After the enzymatic ligation, the double-stranded DNA (dsDNA) is denatured to leave single-stranded DNA (ssDNA). This step may be achieved using DMSO or possibly NaOH. The (ii) and (iii) steps correspond to the first stage of barcode insertion, 370.

The second stage of barcode insertion, 380, involves enzymatic ligation and denaturation. (iv) A second extension sequence including the sequence 356, which is complementary to the second ligation site 360, a second complementary barcode sequence 386, and a capture sequence 388 is hybridized to the second ligation site 360. Then, a primer extension reaction is performed to make a second barcode sequence 390 and the complementary capture sequence 328. (v) Denaturization leaves a ssDNA oligo sequence. The concatenation of the first 350 and second 390 barcodes constitutes the oligo barcode 326 of the LP. A practical method to connect the oligo barcode to the optical barcode of the LP in a large scale is described later.

Alternatively, as shown in FIG. 3C, barcode insertion can also be performed by DNA ligation (T4 DNA ligase). A first extension sequence containing ligation sites 391 and 392, was hybridized to a linker 393. The other part of linker 393 further hybridize to the PCR handle. Therefore, the barcode oligos and PCR handle were brought together by linker 393 and ligated by the T4 DNA ligase. The second stage of barcode insertion can also be performed using DNA ligation. A second extension sequence with ligation site 394 was linked to ligation site 392 via a linker 395.

Although FIGS. 3B and 3C illustrate two-stage extension of two oligo barcodes, the methods can be easily extended to append a third oligo barcode or more oligo barcodes by repeating the ligation steps.

Alternatively, a single LP may be conjugated with multiple different types of oligonucleotide barcode sequences, each with an identical capture sequence and PCR primer, but different ligation sequences. In this case, the combination, not concatenation, of the multiple barcode sequences constitutes the unique oligo barcode of the LP. For example, such LPs can be fabricated by attaching two different types of single-stage oligo sequences (TotalSeq™ B barcodes) to each LP.

Large-scale barcoding provides many uniquely identifiable barcodes, typically in excess of 1,000 or even greater than 100,000. In this context, one may consider that there are a nearly infinite number of barcoding particles having N different types in a pool. From the pool, n particles are taken out in order to tag n cells (or cellular entities) in a sample. The probability of a cell to be uniquely labeled with respect to the rest n -1 cells is given by: P =

$${\left(\frac{N - 1}{N}\right)}^{n - 1} .$$

For large-scale barcoding, that is, N, n > 1000, we find P ≈ e^{-n/N}. The number of uniquely labeled cells is given by: M ≈ N e^{-n/N}. When two or more cells have an identical barcode or identical cellular construct, they may need to be discarded to avoid incorrect identification in the absence of supplemental information. To minimize this duplicate error, it is desirable to have N much greater than n. For N >> n, the duplicate rate is given by

$$1 - {e}^{- n / N} \approx \frac{n}{N} .$$

For example, when N = 10 * n, M ≈ 0.905 N, which indicates that statistically about 10% of the samples would have identical barcodes. To tag over 90% of 10,000 cells uniquely, at least 100,000 uniquely identifiable barcode particles are needed.

Besides triplet microdisk LPs, other multiplet types, such as quartet LPs having four microdisks, may be used with an advantage of the higher number of uniquely identifiable optical barcodes. Also, other types of LPs, such as nanorods and microcubes, may be used, as long as they provide, on a a large-scale, a sufficient number of uniquely identifiable optical barcodes. Other possibilities include microparticles comprising one or more optical resonators operating in a non-lasing regime in which the emission comprises the whispering gallery mode resonances of the resonator, as illustrated in FIG. 2B. In general, microparticles with sizes less than 3 µm in their longest dimension are preferred for applications involving cell tagging. The preferred embodiment shown in FIG. 3A satisfies this condition.

Besides laser-emitting particles, optical barcoding microparticles may be non-laser emitting particles, such as polyyne-based stimulated Raman scattering probes and lanthanide nanophosphors. A combination of these multiplexed particles mixed with different intensity ratios may allow large-scale (1,000 — 100,000) unique optical barcodes.

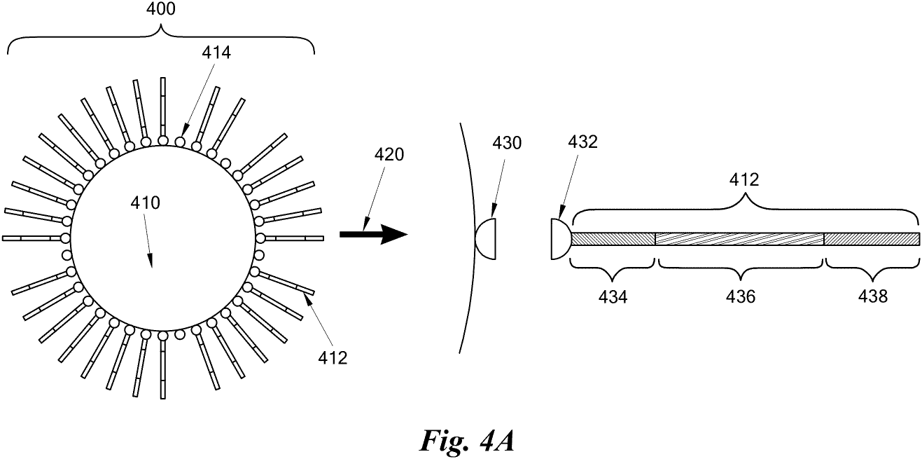

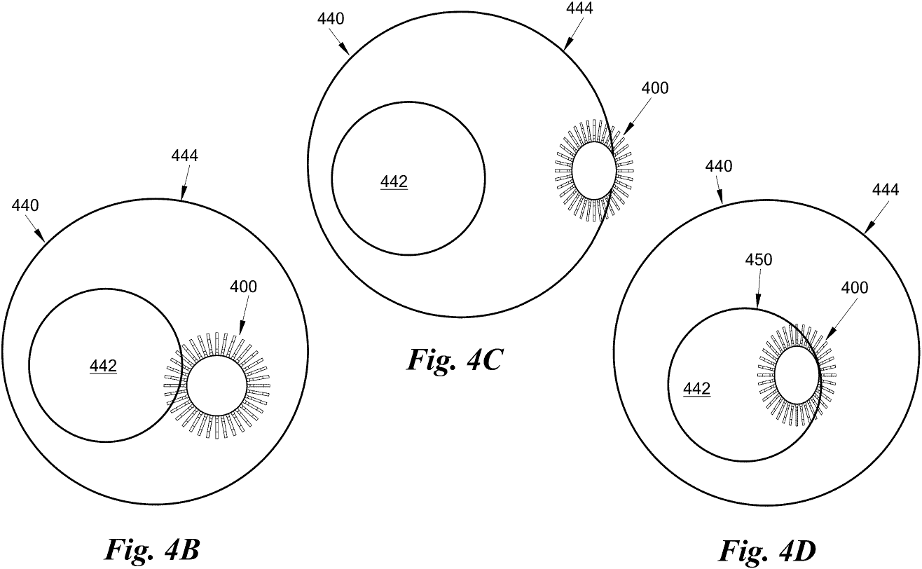

FIGS. 4A through 4D illustrate different ways to tag cells with an oligo-conjugated optical barcoding microparticle 400. One embodiment of the dual-barcoding microparticle 400 has been described above in connection with FIG. 3. Consider an optical microparticle 410 coupled to an oligo-barcoding nucleotide sequence 412. A linker 414 connects the oligo barcode 412 to the microparticle 410. The linker 414 may or may not include a cleavable site. The cleavable site may be a UV-induced cleavable spacer, such as iSpPC or a disulfide bond that can be cleaved by glutathione (GSH). Deoxyribose uracil (dU) can be incorporated to the oligo, and an enzyme mix comprising uracil DNA glycosylase and endonuclease III can be used to cleave the dU site. Upon exposure of UV light (indicated by arrow 420) having appropriate spectral content (300-350 nm) and intensity, the photocleavable spacer is cleaved into two pieces 430 and 432, dissociating the oligo sequence 412 from the microparticle. As shown in FIG. 4A, the oligo sequence includes a PCR primer 434, a microparticle-associated oligonucleotide barcode 436, and a complementary capture sequence 438.

The multi-barcoding particle 400 can be used to tag cellular entities. FIG. 4B shows an example in which a cell 440 with a nucleus 442 has internalized the particle 400. This intracellular tagging can be performed using such processes as macropinocytosis, endocytosis, and fusion liposomal delivery through a cellular membrane 444. To facilitate the intracellular uptake, the particle 400 may be further coated with cationic lipids or positively charged polymers, such as polylysine or polyethylenimine (PEI).

FIG. 4C depicts another example in which the multi-barcoding microparticle is attached to the external surface of the cell membrane 444. For this extracellular tagging, the surface of the microparticle 400 may be coated with membrane binding molecules, such as antibodies targeting specific surface proteins abundant in the target cell 440, lipids that can anchor on the cellular membrane 444, or molecules with the N-hydroxysuccinimide (NHS) group that can bind to the amine group of cell membrane proteins.

FIG. 4D illustrates yet another example in which the microparticle 400 is bound to the nuclear membrane 400. This nuclear tagging is useful for single-nucleus RNA sequencing or for a single-nucleus assay for transposase-accessible chromatin sequencing (ATAC-seq).

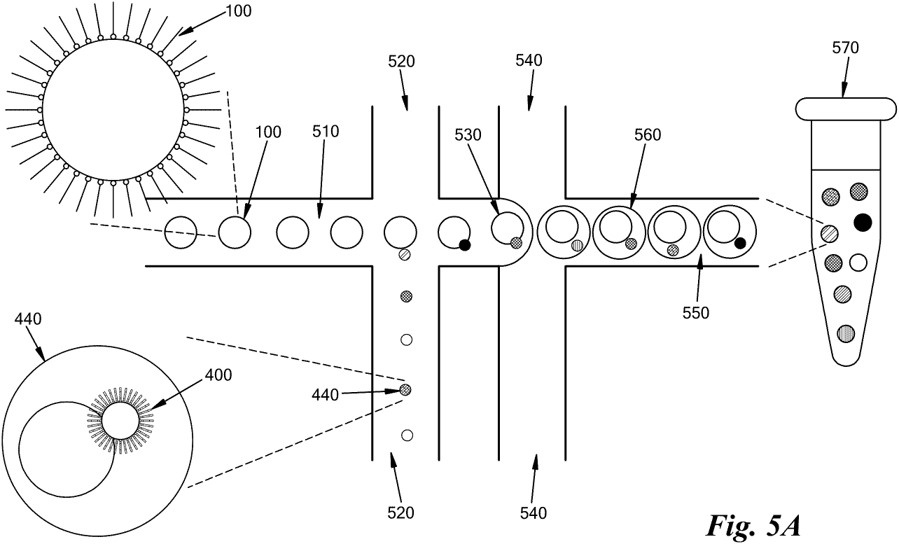

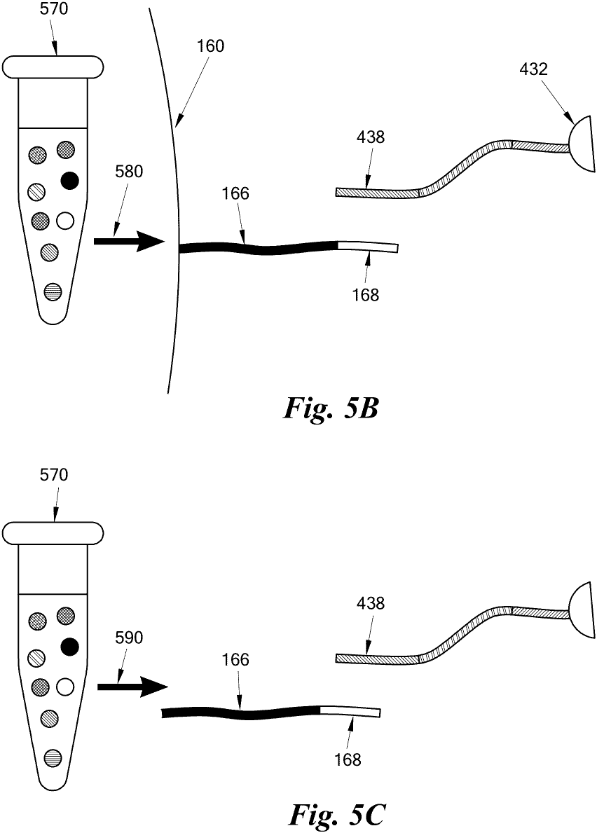

FIGS. 5A through 5C illustrate a method for the construction of a cDNA library for single-cell sequencing of cells tagged with dual-barcoding microparticles. FIG. 5A depicts a typical microfluidic device for encapsulating the oligo-barcoded microbead 100 and the cell 440 tagged with the multi-barcoding microparticle 400 into a droplet. Oligo-barcoded microbeads are flowed through a first input flow channel 510, and cells are flown through a second input channel 520, which intersects with the first input channel 510. A pair of an oligo-barcoded microbead and a single cell is incorporated into a droplet by pinching with oil caused to flow in a third channel 540, which also intersects with the first channel 510. In an output channel 550, the generated droplets 560 are collected into a vial 570. This step is called cell portioning.

Various steps are then performed to produce the cDNA library from the droplets. In conventional droplet-based sequencing, such as Drop-seq and 10X Genomics, the workflow steps involve cell lysis, mRNA capture, reverse transcription, breaking emulsion, cDNA cleanup, cDNA amplification, and constructing the library, prior to high-throughput next generation sequencing, such as Illumina sequencing. After cellular lysis, both intracellular mRNAs and the oligo barcode of the microparticle 400 are captured by the capture sequence of the oligo-barcoded bead 100 and are indexed via reverse transcription.

In the 10x Genomics Single Cell 3′ v3 assay, once cells in a sample are partitioned, the gel beads are dissolved, and their oligo primers are released into the aqueous environment of the droplet. The contents of the droplet including oligos, lysed cell components and master mix are incubated in a reverse transcription reaction to generate full-length, barcoded cDNA from the poly A-tailed mRNA transcripts. The reverse transcription reaction is primed by the barcoded gel bead oligo, and the reverse transcriptase incorporates the template switch oligo via a template switching reaction at the 5′ end of the transcript. The droplets are then broken, pooling single-stranded, barcoded cDNA molecules from every cell. Bulk PCR-amplification and enzymatic fragmentation are then performed. Size selection is used to optimize the insert size of the double-stranded cDNA prior to library construction. During library construction a Read-2 sequence is added by adapter ligation. Illumina P5 and P7 sequences and sample index sequences are added during the sample index PCR. The final library fragments contain P5, P7, Read-1, and Read-2 sequences used in Illumina bridge amplification and sequencing. Additionally, each fragment contains the 10x barcode, UMI and cDNA insert sequence used in data analysis.

In embodiments of the present invention, almost all the above mentioned 10X workflow steps are also applied. An additional step may be included to release the oligonucleotide sequence attached on the multi-barcoding microparticle 400. FIG. 5B illustrates this process 580, which allows the complementary capture sequence 438 in the released oligonucleotide sequence to be captured by the capture sequence 168 in the oligo-barcoded microbead 160. The microbead 160 may typically employ its own, photocleavable or chemically cleavable spacer. In this case, UV illumination or the presence of chemical or enzymatical cleaving reagent in the droplet solution causes release of the capture sequence. This process 590 facilitates the binding of capture sequences 168 and 438. The free-floating hybridized oligonucleotide sequences are converted to dsDNA via reverse transcription and amplified by PCR.

Especially when the microbead 110 release its oligonucleotide sequence, the linker 414 may not need to be cleavable. The hybridized oligonucleotide sequences 158 and 438 on the surface of a multi-barcoding particle can be converted to dsDNA via reverse transcription. And the product may be spontaneously released from the microparticle into the surrounding fluid during cDNA cleaning and enrichment and then later amplified by PCR.

In a manner similar to the CITE-seq workflow, the amplified cDNAs of the mRNA and the microparticle-associated molecular barcodes can be separated according to their different sizes, and the two libraries can be sequenced together or separately in Illumina sequencing. The single-cell transcriptomics data and molecular barcode data are then aligned according to the oligo barcode on the barcoded microbead 110.

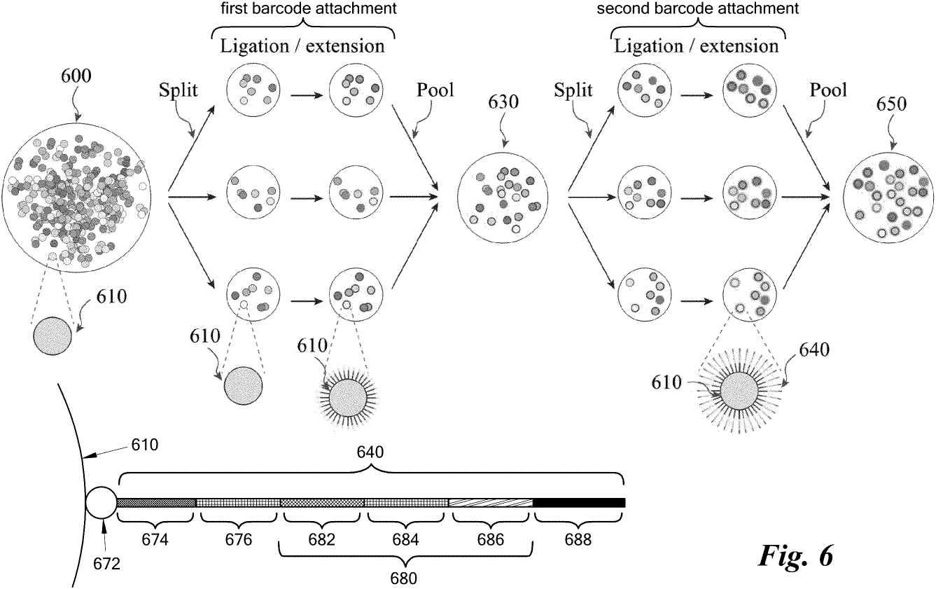

It is necessary, or at least preferable, to know the association of the optical and molecular barcodes of each multi-barcoding microparticle. FIG. 6 illustrates a fabrication method to accomplish this result. It is based on a modified split-pool technique. Each microparticle is tracked during each splitting or pooling process by measuring its optical barcode. A large number of optical microparticles 600, such as multiplet LPs, with a sufficient number of distinctive optical barcodes are prepared. Each microparticle 610 in the pool may be coated with identical adapters and PCR handles. Alternatively, adapters and PCR handles may be attached later after splitting along with first barcodes.

The microparticles 600 are split into different wells in a multi-well plate with approximately equal numbers of microparticles per well. Standard 96-, 384-, or 1536-well plates may be used. Then, distinctively different, first oligo barcodes are administered to different wells and attached to the microparticles via hybridization and extension. Microparticles in the same well are given the same first oligo barcode, and microparticles in distinct wells are given distinct first oligo barcodes. A liquid handler may be used to facilitate the ligation and enzymatic elongation process. This process is similar to the method used for fabricating InDrop barcoded beads¹¹.

Either during or after splitting, an appropriate optical setup employing an optical barcode reader is used to measure and record the optical barcodes of all the microparticles in each well. Particularly when LPs are used as microparticles, the optical reader may be implemented by a pump light source and a spectrometer. The pump light source may be a continuous-wave laser or nanosecond pulsed laser. The spectrometer may implemented by a diffraction grating and a line scan camera, but other configurations known in the art can be used. Preferably the spectral resolution of the spectrometer is in the order of 1 nm. The optical reader may be coupled to an imaging setup or microscope, and the microparticles are scanned either using a translation sample stage or an optical beam scanner. Alternatively, the optical reader may be coupled to a flow or microfluidic setup, wherein the microparticles are scanned as they are flowing in a fluidic stream.

As one embodiment of such an optical barcode scanning setup, we have modified a capillary-based commercial flow cytometer (Guava easyCyte™, Luminex) by connecting a nanosecond ytterbium-doped fiber laser (a center wavelength of 1030-1065 nm, pulse duration of 5-20 ns, repetition rate of 1-5 MHz) and a grating spectrometer with a diffraction grating and an InGaAs line scan camera. Microparticles are aspirated from a vial using the capillary tubing of the cytometer. As the particles pass through the pump beam illuminating the capillary, their emission spectra are measured by the spectrometer. The typicalmeasurement rate is about 1,000 particles per second. After measurement, a desired number of particles is dispensed into each well of a multi-well plate by reversing the flow after holding the microparticle in a reservoir. Instead of the capillary-based setup, a flow cell employing hydrodynamic focusing using sheath fluid may be used, with an advantage of a higher acquisition rate, for example, up to 20,000 events per second.

After adding the first barcode sequence, the microparticles in the wells are pooled into a single vial 630. Then, in the second stage the microparticles are split again into multiple wells, and second oligo barcode sequences, different for different wells, are added and attached to the microparticles via ligation. This forms an oligo sequence 640 containing the first and second barcode sequence (more precisely, the conjugate sequences of the first and second barcode sequences are incorporated into the microparticles, as depicted in FIG. 3B). Finally, the microparticles in the multiple wells are pooled into a vial 650. The oligo sequence 640 may include some or all of the following elements: a linker 672, a PCR handle 674, a first ligation site 676, the first barcode sequence 682, a second ligation site 684, the second barcode sequence 686, and complementary capture sequence 688. The concatenated first and second barcode sequences in the central region 680 represent the microparticle-specific molecular barcode.

An exemplary sequence compatible with 10X single cell 3′ v3 is as follows: /5AmMC12 (conjugation linker and spacer)/[GTGACTGGAGTTCAGACGTGTGCTCTTCCGATCTNNNNNN] (PCR handle and ligation site) / [NNNNNNN] (first barcode) / [NNNNNNNNNNNNN] (second ligation site) [NNNNNNN] (second barcode) / [GCTTTAAGGCCGGTCCTAGC*A*A] (complementary capture sequence) (SEQ ID NO: 1). Here, [N] represents a random nucleoside, one of [A], [C], [G], and [T], [B] represents either [C], [G], or [T], and asterisk (*) indicates a phosphorothioated bond that is used to prevent nuclease degradation. In this example, the PCR handler 674 also serves the role of the first ligation site.

Although FIG. 6 describes a two-stage split-pool process, the method can be easily extended to a three-stage split-pool or more stages. For example, we can use two 96-well plates in each stage to make 192 × 192 × 192 = 7,077,888 different combinations of oligo barcodes.

It is desirable for the vast majority or all of the microparticles in a final pool or vial to be unique — with unique identification represented by the combination of their optical and oligo barcodes. It is not necessary for the unique microparticles to have both unique optical barcodes and unique oligo barcodes. For example, we may begin the oligo conjugation process with ~700,000 triplet microdisk LPs in a starting pool. They are split into 192 wells in each stage about 3,600 to 3,700 LPs per well on average. After the three-stage split-pool process, in the final pool, about 90% LPs have unique oligo barcodes. However, almost all LPs in the final pool would not have unique optical barcodes because the total number of unique barcodes of triplet LPs is much less than the total number of LPs. This is not always a problem when only a small fraction of the LPs is used for a given sample or a population of cells. For example, suppose we take only 20,000 LPs from the final pool. It is highly likely that all the LPs in this population of 20,000 LPs have both unique oligo barcodes and unique optical barcodes. Then, they are all distinguishable from each other within the population.

The number of oligo barcodes on each LP typically should be optimized. Too many microparticle-associated oligo barcodes may compete with mRNAs to bind to the beads and overwhelm the sequencing step when the poly(dT) capture sequence is used for capturing the microparticle-specific oligo sequence. On the other hand, too few oligo barcodes will make the detection difficult. The possible number of unique molecular identifier (UMI) copies may range from 100 to 10,000, and an optimum copy number may be approximately 1,000 copies per microparticle. When feature barcodes that are not poly(dA) are used, the number of barcodes on each microparticles may be less of a concern since there is no direct competition between the oligo barcodes with mRNAs. Nonetheless, the number of barcodes released from microparticles may be chosen to be within a range of 100 to 100,000.

We have fabricated prototype barcoded microparticles according to the design in FIG. 3B. After establishing the protocol with silica-coated beads, we used silica-coated, InGaAsP single-microdisk (singlet) LPs with diameters of about 2 µm. The laser microparticles were functionalized with a PCR handle using carbodiimide crosslinking chemistry. Using the method described in FIG. 3B, a sequence /SAmMCI12/[GCTAGTTC][CCTTGGCACCCGAGAATTCC][CACTGAA][CTCATCGCA TTCGCTC][ACGTCGAT][BAAAAAAAAAAAAAAAAAAAAAAAAAAAAAA*A*A] (SEQ ID NO: 2) was conjugated to the carboxyl group in the silica surface. Then, a 10× v3 capture sequence, CTACACGACGCTCTTCCGATCTAAACCTGAGAAACCGCCTGTTCGTATCGTTTTT TTTTTTTTTTTTTTTTTTTTTTTTT (SEQ ID NO: 3), was added to the medium and used as the reverse primer for enzymatic extension (5′ to 3′). The capture sequence and a forward primer, CCTTGGCACCCGAGAATTCC (SEQ ID NO: 4), were used of PCR amplification. In 50 µL reaction, we used 25 µL of TaqMaster Mix, 1 µL of forward primer (20 µM stock), 1 µL of 10× v3 capture sequence (20 µM stock), and 23 µL of barcoded microparticles in nuclear-free H₂O. The final product is dsDNA with 134 bp

(CCTTGGCACCCGAGAATTCCCACTGAACTCATCGCATTCGCTCACGTCG
ATBAAAAAAAAAAAAAAAAAAAAAAAAAAAAAAAACGATACGAACAGGCG
GTTTCTCAGGTTTAGATCGGAAGAGCGTCGTGTAG (SEQ ID NO: 5)
).

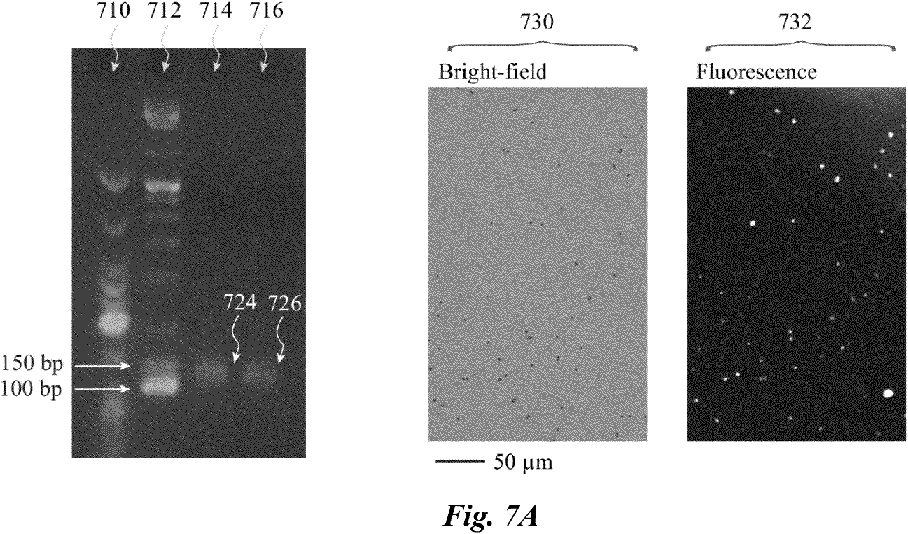

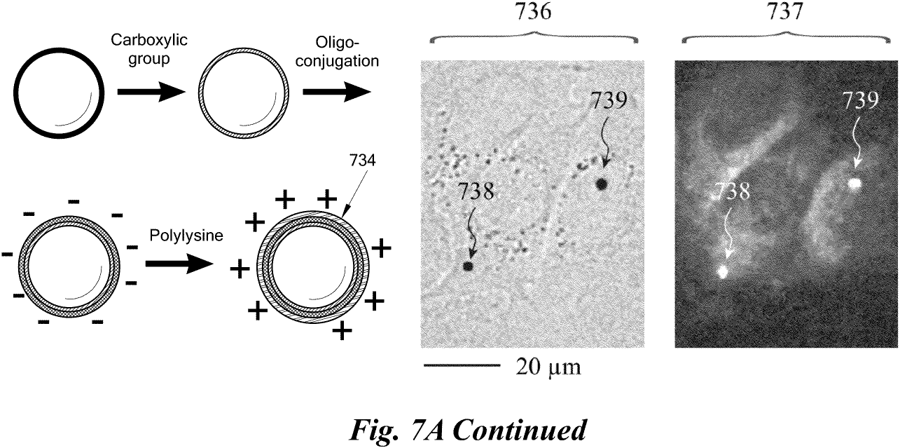

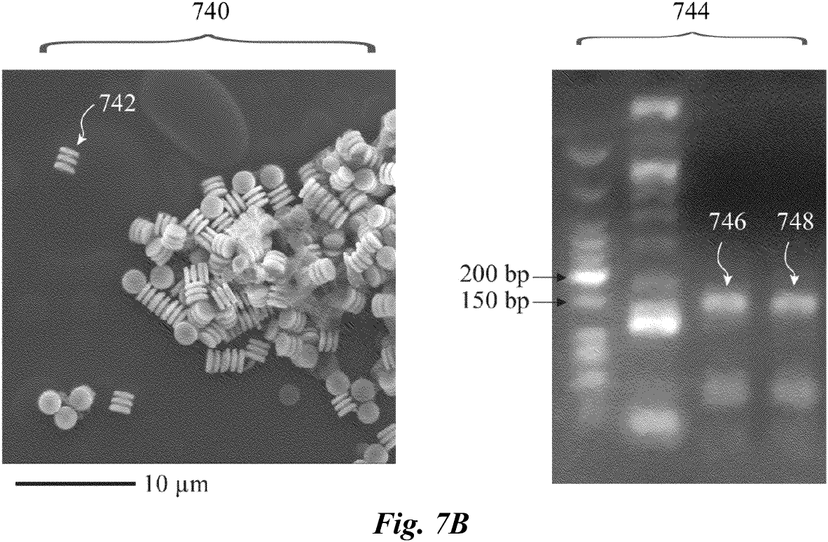

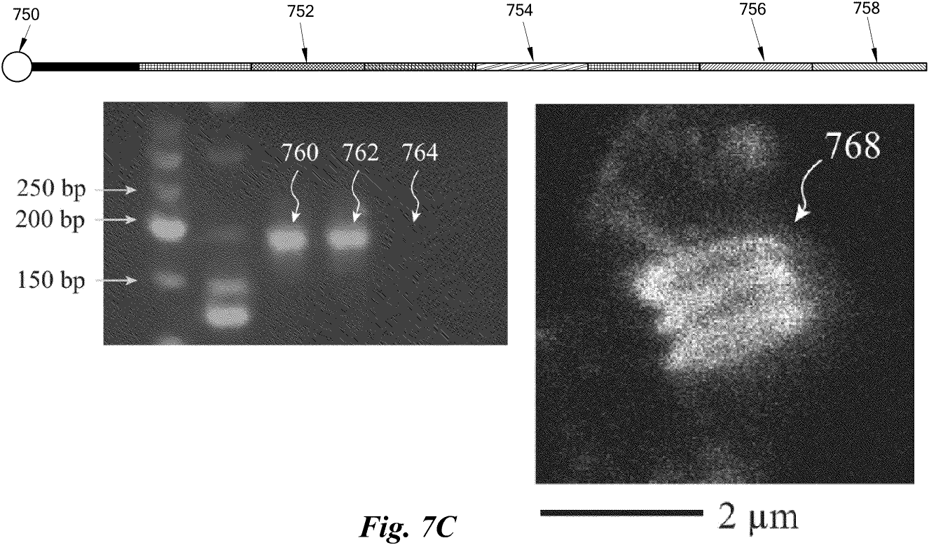

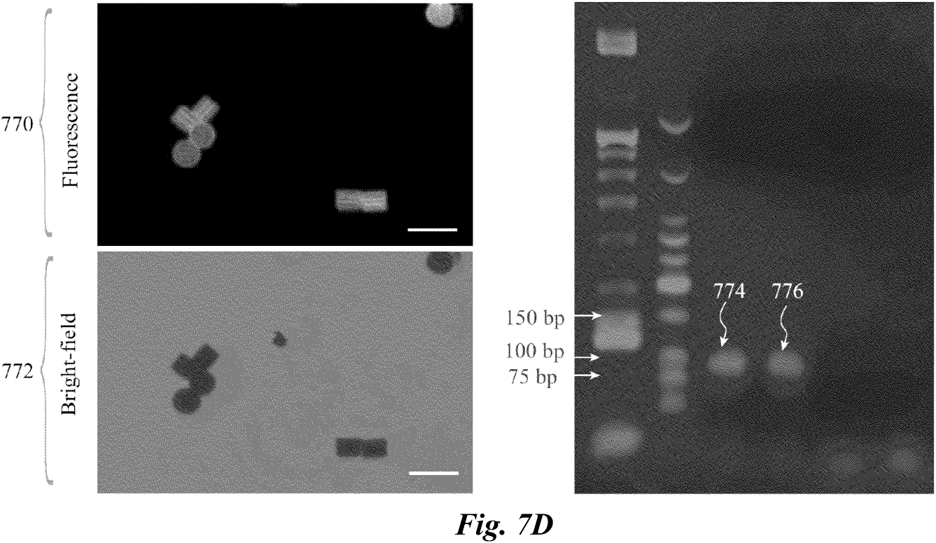

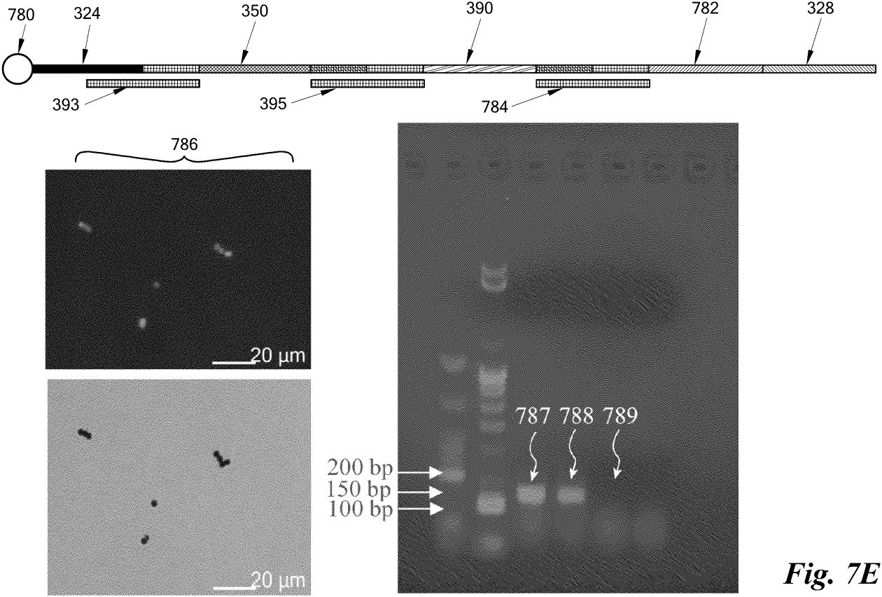

FIG. 7A shows the experimental results. In the gel electrophoresis image of the PCR product, the first two columns, 710 and 712, are for standard DNA ladder samples, showing the positions of 100 bp and 150 bp. The next two columns, 714 and 716, are obtained from the oligo-coated LP samples, which show 134 bp bands, 724 and 726. These results confirm the presence of the oligo barcode sequence on the surface of the LPs. FIG. 7A also shows bright-field images 730 and fluorescence images 732 of the oligo-attached LPs in the well plates after adding a FISH probe, 5′-/6-FAM/VTTTTTTTTTTTTTTTTTTT-3′ (SEQ ID NO: 6), which is a single isomer derivative of fluorescein dye, 5′6-FAM, and poly(dT). The oligo-coated LPs show fluorescence signals indicating the presence of the oligo sequence, whereas control LPs, having an uncoated silica surface, showed no fluorescence signal.

Oligo-barcoded microparticles can be used to tag cells by cellular internalization or physical or chemical attachment on the cell membrane. We found that the tagging time and efficiency can be enhanced by encapsulating the microparticles with appropriate functional molecules, such as polylysine 734. Polylysine binds to negatively charged oligonucleotides, forms a positively charged, protective layer. The positively charged polymer can facilitate the association of LPs with negatively charged cellular membrane.

To test in vitro stability, 4T1 cells in culture plates were incubated with oligobarcoding LPs with polylysine coating. After incubation with LPs for 24 hours, the cells were fixed, permeabilized, and washed. Afterwards, the 5′6-FAM-dT FISH probe was added to the fixed cell. Bright-field 736 and fluorescence 737 images of the sample show dual-barcoding LPs 738, 739 in the cytoplasm and bright fluorescence signals from the microparticles. The FISH probes were added and measured after incubating the cells with the microparticles for 24 hours. This shows the stability of the oligo barcodes on the microparticles in the cytoplasm.

To further enhance the stability of the oligo barcodes in cellular entities as well as tissues and fluids surrounding cellular entities, threose nucleic acid (TNA)-based oligos may be used instead of DNA-based oligos, described above. TNA is an artificial genetic polymer, which can base pair with complementary sequences of DNA and RNA. Unlike DNA, TNA is refractory to nuclease digestion. One method to incorporate TNA oligo barcodes to LPs is to attach a first DNA segment as depicted in FIG. 3B (i), and then attach a TNA-based oligo segment including a first TNA oligo barcode using a TNA polymerase in a process analogous to that depicted in FIG. 3B (ii-iii). The second and third TNA oligo barcodes can be concatenated using this transcription process.

FIG. 7B shows another set of experimental results on dual-barcoding microparticles 740 based on triplet LPs 742. The triplet LPs were coated with oligo barcodes by using the processes depicted in FIG. 3B. In this experiment, the first sequence conjugated to triplet LPs was [NH2]-[GTGACTGGAGTTCAGACGTGTGCTCT][TCCGATCTAAGATTGCAC] (SEQ ID NO: 7). According to FIG. 3B, the linker 330 was NH2, the primer 324 was GTGACTGGAGTTCAGACGTGTGCTCT (SEQ ID NO: 8), and the ligation site 340 was TCCGATCTAAGATTGCAC (SEQ ID NO: 9). The 2^{nd} oligo segment we used was [CAACATCAGATGCTCA][NNNNNNNNNNNNNNN][GTGCAATCTTAGATCGGA] (SEQ ID NO: 10), which is a concatenation of 356, a barcode 346, and 336. After ligation, this segment adds the first oligo barcode 350, which is the conjugate sequence of 346. The 3^{rd} oligo piece used was [TTGCTAGGACCGGCCTTAAAGC][NNNNNNNNNNNNNN][CAACATCAGATGCTC A] (SEQ ID NO: 11), which corresponds to a concatenation of 388, 386, and 356. After ligation, the final sequence is NH₂-GTGACTGGAGTTCAGACGTGTGCTCTTCCGATCTAAGATTGCACNNNNNNNNNN NNNNGAGCATCTGATGTTGNNNNNNNNNNNNNNGCTTTAAGGCCGGTCCTAGC AA (SEQ ID NO: 12). The bead capture sequence was [GTCAGATGTGTATAAGAGACAGAAACCTGAGAAACCGCCTGTTCGTATCG[TTG CTAGGACCGGCCTTAAAGC] (SEQ ID NO: 13). The final PCR using GTGACTGGAGTTCAGACGTGTGCTCTTCCGATCT (SEQ ID NO: 14) as the forward primer and GTCAGATGTGTATAAGAGACAG (SEQ ID NO: 15) as the reverse primer results in a 161-nt sequence: [GTGACTGGAGTTCAGACGTGTGCTCTTCCGATCT][AAGATTGCAC][NNNNNNNN NNNNNNN][TGAGCATCTGATGTTG][NNNNNNNNNNNNNN][GCTTTAAGGCCGGT CCTAGCAACGATACGAACAGGCGGTTTCTCAGGTTT][CTGTCTCTTATACACATC TGAC] (SEQ ID NO: 16). A gel electrophoresis image 744 confirms the presence of the final 161-bp PCR product in two samples 746 and 748.

FIG. 7C shows yet another example, wherein three-stage oligo barcode sequences were attached to triplet LPs using three-stage ligation extension. The final oligo sequence includes a linker 750, the first piece of barcode 752, the second piece of barcode 754, and third piece of barcode 756, as well as a capture sequence 758. The electrophoresis image of a 20-cycle PCR product showed the 195 bp band from two LP samples 760, 762, whereas control (supernatant) did not show a 195 bp band 764. The presence of the oligo barcodes on LPs was confirmed by FISH imaging. Fluorescence images of a triplet particle 768 with a FISH probe hybridizing to the capture sequence attached confirms a successful coating of the three-stage oligo barcode. The total read length was 90 bp, sufficient to read the 89 bp-long three-stage barcode including ligation sites between barcoding sequences.

In another demonstration as shown in FIG. 7D, using an NHS ester crosslinker DTSP, a PCR handle sequence /5AMC12/GTGACTGOAGTTCAGACGTGTGOTCTTCCGATCT (SEQ ID NO: 17) was conjugated to the surface of triplet-LPs. The conjugated DNA can be measured by fluorescence in situ hybridization (FISH) and imaged under a microscope, as a complimentary sequence hybridizes to the conjugated DNAs and emits fluorescence. Successful conjugation of DNA oligos to the LP is achieved. In addition, zeta potential of the silica surface in each step of modification was also measured for the DTSP method, showing successful conjugation of negatively charged DNA on LPs. The triplet LPs were coated with oligo barcodes by using the processes depicted in FIG. 3C. In this experiment, the first sequence 324 conjugated to triplet LPs was /5AmMC12/GTGACTGGAGTTCAGACGTGTGCTCTTCCGATCT (SEQ ID NO: 17). The 2^{nd} oligo segment we used was /5Phos/ACATGGNNNNNNNNTATCTAC (SEQ ID NO: 18). The 2^{nd} oligo contains ligation site 391 (ACATGG), barcode 350 (NNNNNNNN), and ligation site 392 (TATCTAC), with linker 393 (CCATGTAGATCGGAAGAGCA (SEQ ID NO: 19)). After ligation, this segment adds the first oligo barcode 350. The 3^{rd} oligo piece used was /5Phos/GTCACGNNNNNNNGCTTTAAGGCCGGTCCTAGC * A * A (SEQ ID NO: 20). The 3^{rd} oligo contains ligation site 394 (GTCACG), barcode 390 (NNNNNNN), and capture sequence 328, with linker 395 (CGTGACGTAGATA (SEQ ID NO: 21)). This process results in a 90-nt final sequence: /5AmMC12/GTGACTGGAGTTCAGACGTGTGCTCTTCCGATCTACATGGNNNNNNN NTATCTACGTCACGNNNNNNNGCTTTAAGGCCGGTCCTAGC*A*A (SEQ ID NO: 22), which is the same length as the Totalseq B sequence. The presence of the oligo barcodes on LPs was confirmed by FISH imaging. Bight fluorescence (770) from triplet particles with a FISH probe hybridizing to the capture sequence was observed. Using GTGACTGGAGTTCAGACGTGTGCT (SEQ ID NO: 23) as the forward primer and TTGCTAGGACCGGCCTTAAA (SEQ ID NO: 24) as the reverse primer, the gel electrophoresis image of a 20-cycle PCR product showed the 90 bp band from two LP samples 774, 776, whereas control (supernatant) did not show a 90 bp band 778. Furthermore, we quantified the number of oligos using qPCR, which is ~10⁵ /particle. To test the feasibility of the split-pool method described in connection with FIG. 6, we used a population of LPs and appended 4 different first barcodes in the first stage of split-pool and then another 4 different second barcodes in the second stage of split-pool. This process produced a total 16 different two-stage oligo barcodes on LPs. We performed bulk sequencing of the PCR products obtained from the microparticles and confirmed all 16 oligo barcodes.. We sequenced for 1 million reads. The sequencing results all matched with the expected results (correct reads > 92 % of total reads).

FIG. 7E shows another example, wherein three-stage oligo barcode sequences were attached to triplet LPs 780 using T4 DNA ligase depicted in FIG. 3C. The first sequence was conjugated to triplet LPs (PCR handle, 5AmMC12/GTGACTGGAGTTCAGACGTGTGCTCTTCCGATCT (SEQ ID NO: 17)). The 2^{nd} oligo segment was /5Phos/ACATGGNNNNNNNNTATCTAC (SEQ ID NO: 18), with linker 393 (CCATGTAGATCGGAAGAGCA (SEQ ID NO: 19)). The 3^{rd} oligo piece used was /5Phos/ GTCACGNNNNNNNGATGAAT (SEQ ID NO: 25), with linker 395 (CGTGACGTAGATA (SEQ ID NO: 21)) The 4^{th} oligo piece used was ACGGCGNNNNNNNGCTTTAAGGCCGGTCCTAGC*A*A (SEQ ID NO: 26), with linker 784 (CGCCGT ATTCATC (SEQ ID NO: 27)). The total oligo length was 109 bp, with a sequence GTGACTGGAGTTCAGACGTGTGCTCTTCCGATCTACATGGNNNNNNNTATCTAC GTCACGNNNNNNNGATGAATACGGCGNNNNNNNGCTTTAAGGCCGGTCCTAGC* A*A (SEQ ID NO: 28). Fluorescence images 786 of a triplet particles with a FISH probe hybridizing to the capture sequence attached confirms a successful coating of the three-stage oligo barcode. The bead capture sequence was GTCAGATGTGTATAAGAGACAGAAACCTGAGAAACCGCCTGTTCGTATCGTTGC TAGGACCGGCCTTAAAGC (SEQ ID NO: 13). The final PCR using GTGACTGGAGTTCAGACGTGTGCT (SEQ ID NO: 23) as the forward primer and GTCAGATGTGTATAAGAGACAG (SEQ ID NO: 15) as the reverse primer results in a 159-nt sequence: GTGACTGGAGTTCAGACGTGTGCTCTTCCGATCTACATGGNNNNNNNTATCTAC GTCACGNNNNNNNGATGAATACGGCGNNNNNNNGCTTTAAGGCCGGTCCTAGC AA CGATACGAACAGGCGGTTTCTCAGGTTTCTGTCTCTTATACACATCTGAC (SEQ ID NO: 29). A 20-cycle PCR product showed the 159 bp band from two LP samples 787, 788, whereas control (supernatant 789) did not show the band.

To produce a larger number of uniquely barcoded microparticles, we could use a 384-by-384 split-pool process using two 384 well plates. Alternatively, we could use a 192-by-192-by-192 split-pool process, using two 96-well plates in each of 3 stages of conjugation. Using quartet LPs and the 3-stage oligo conjugation method, it should be possible to produce a larger number, greater than 100,000 of uniquely dual-barcoding microparticles.

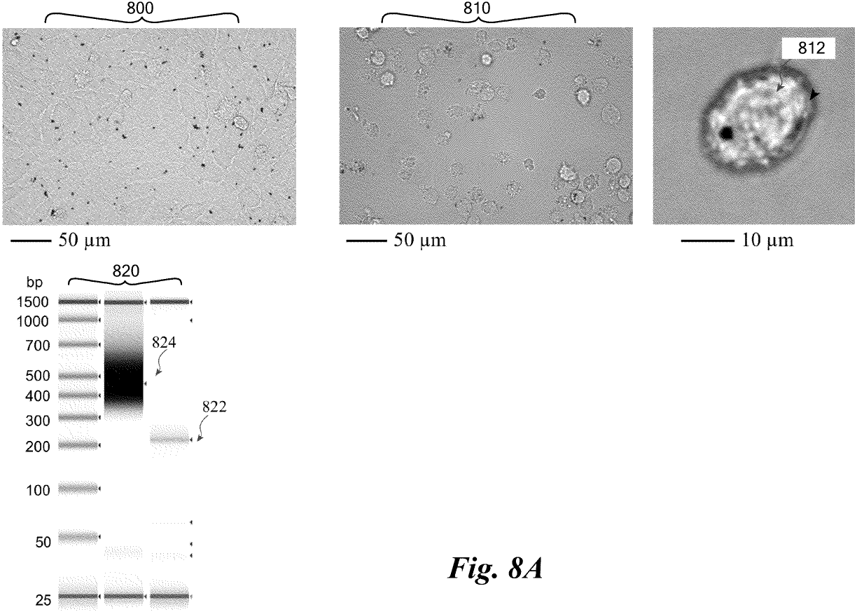

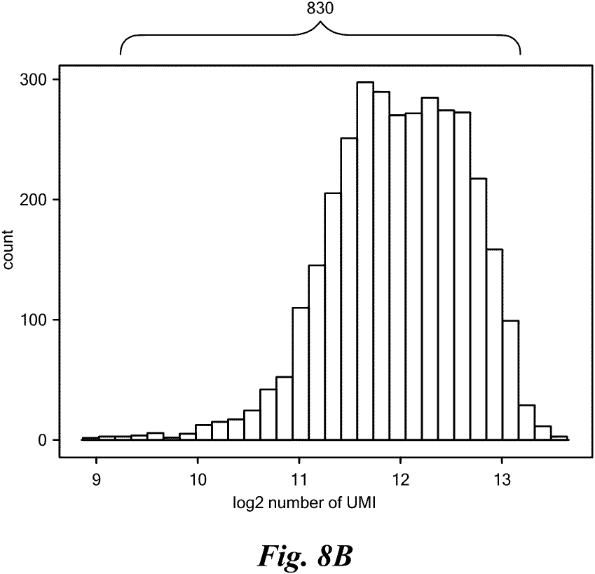

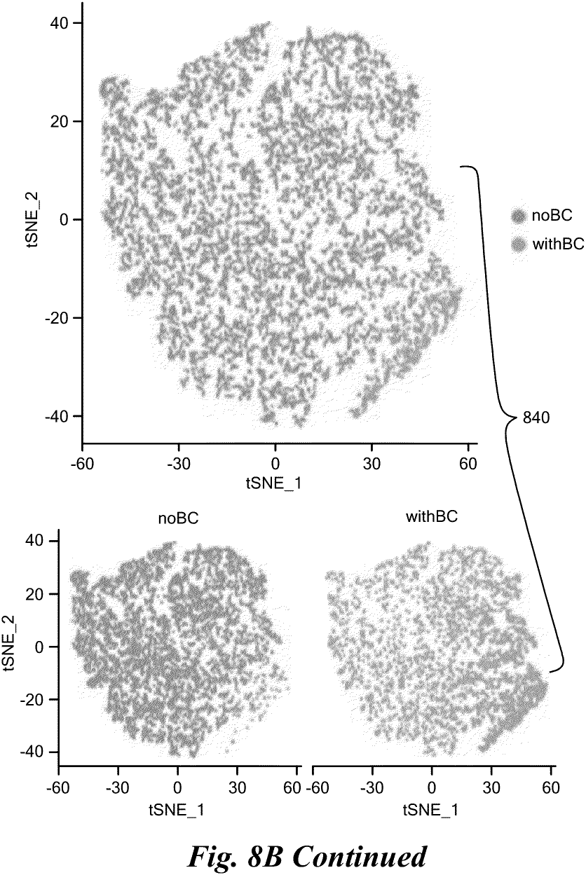

FIG. 8A shows experimental results obtained with these dual-barcoding LPs. HeLa cells 800 internalized the microparticles after incubation for 24 hours. The number of LPs per cell varies from zero to several particles. When more than one LP is in a cell, the collective optical emission spectra and oligo barcode sequences constitute the identification data of the particular cell. After trypsinization, the cells 810 maintained their associated microparticles. A single cell 812 containing a single dual-barcoded LP 814 is shown. To obtain single-cell transcriptomic information, a 10X Genomics Chromium Controller instrument was used to produce the sequencing libraries for both the oligo sequences on the LPs as well as mRNA in the cells. We tested our workflow using the 10x Chromium Single Cell 3′ v3.1 chemistry with feature barcoding technology. The dual-barcoded LPs were introduced to Hela cells by incubation with cells for 24 h. The LP-tagged cells were dissociated, subjected to droplet-based platforms, and encapsulated into nanoliter-sized droplets. Totally ~10,000 cells were analyzed. After cell lysis, both the mRNAs in the cell and the LP barcodes were captured and indexed by cell barcodes via reverse transcription to form cDNAs. The cDNAs were separated based on their size differences, amplified, and the two libraries were separately prepared and sequenced together in Illumina-sequencing. An electrophoresis image 820 shows a band above 200 bp (822) as expected for the feature-barcode library. By contrast, the mRNA library 824 has a typical band ranging from 300 to 1000 bp.

We fabricated dual-barcoded LPs by two rounds of split-pool for a small-scale 4×4 design (totally 16 LP-barcode types). We sequenced around 10,000 read pairs per cell for gene expression library and 2,500 for LP-barcode library using the Illumina NextSeq 2000 in the Sequencing Core of our institution. As shown in FIG. 8B, our result confirmed that the LP barcodes can be successfully captured, reverse-transcribed to cDNAs, amplified and sequenced. In addition, over 90% of the reads (out of total) in the LP-barcode library correctly matched with the theoretical sequences. The LP barcodes were correlated with valid cell barcodes with good signal (number of UMIs > 200, as shown in histogram 830). The background LP-barcode signals can be identified and easily differentiated from the real signals in data analysis. Our sequencing result showed 3363 HeLa cells contain LP barcodes, while 6170 HeLa cells don’t, which agrees well with our microscopic observations that 30-40% of Hela cells were tagged with LPs. In addition, no obvious perturbation of cell transcriptome was observed in the tSNE graph 840 after tagging with LP-barcodes.

These experiments demonstrate the feasibility of generating a cellular coding construct that uniquely codes a cell, or more broadly a cellular entity, wherein the cellular coding construct comprises at least one laser particle and a structurally coded oligonucleotide sequence. The structurally coded oligonucleotide and the laser particle have a physical association (in these examples, chemically conjugated). They are configured for physical association with the cellular entity and also configured for distinctive identification of the cellular entity.

Beside the dual-barcoding microparticle, the cellular construct can further comprise a non-volatile storage arrangement encoded with identification data characterizing the structurally coded oligonucleotide and the laser particle. Such identification data include the lasing wavelengths (WL’s) of the laser particle and the oligo sequences of the barcodes attached to the laser particle. The identification data may further include other information, such as intensity or power (P’s) of laser emission peaks, a production lot number (LOT), and an identifier (ID) number representing the particular dual-barcoding particle.

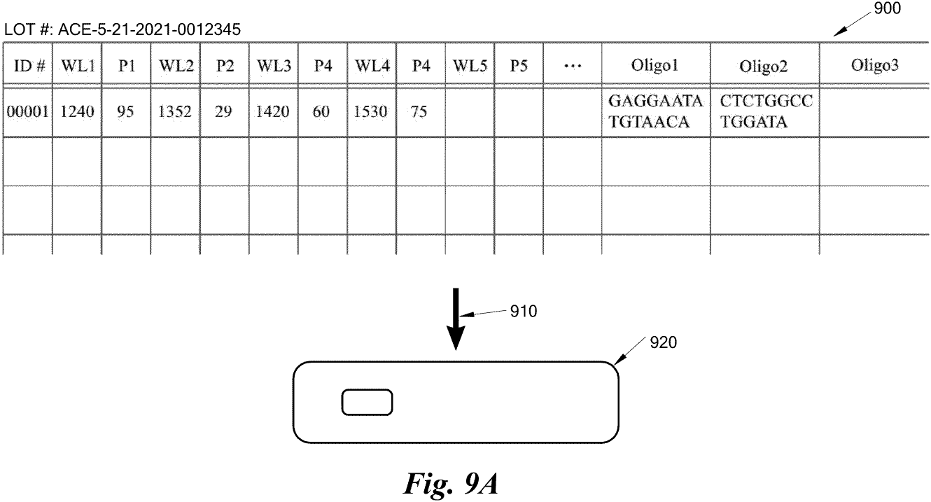

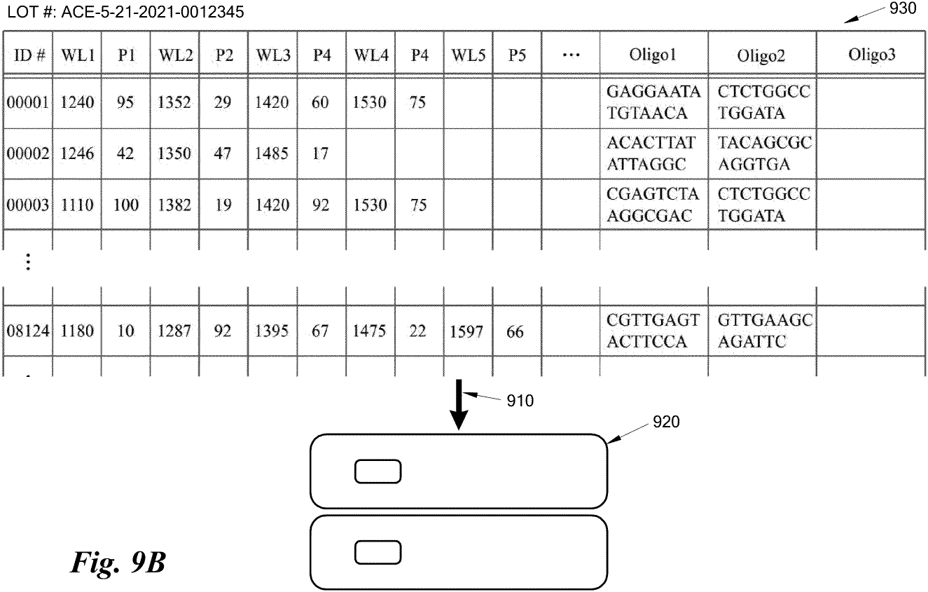

FIG. 9A illustrates this preferred embodiment. The identification data 900 in the form of a table is illustrated. The data 910 are stored, or possibly scribed, in a non-volatile storage arrangement 920. The storage arrangement may be implemented with structures including a semiconductor memory chip, magnetic hard disk, optical disk.

To tag a large number of cells or cellular entities, a population of objects, wherein each object is a distinct cellular construct may be used. In this case, it is convenient to have all the identification data of the population of cellular constructs in a single storage arrangement. FIG. 9B illustrates this embodiment. A list 930 of identification data of the cellular constructs is stored into the storage arrangement 920. Although we illustrate the identification data in a tabular form, the data can be stored in various formats including binary or ASCII files.

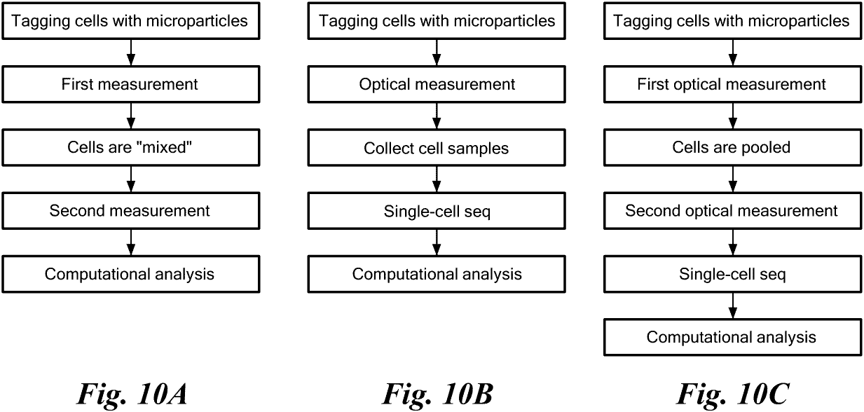

FIG. 10A depicts a general workflow of cell tracking-based analysis enabled by the multi-barcoding microparticles. Cells can be analyzed using one method to acquire a specific set of information and then moved to another instrument acquiring the second set of information. Subsequently, the two sets of information can be combined and aligned to individual cells based on their barcodes identified in each measurement step.

In this workflow, an initial step is to establish physical associations between microparticles and cells. The associations are achieved typically by using chemical bonding, such as protein-protein interaction between the cell membrane and the surface coating material of the laser particles or the encapsulation of microparticles in the cytoplasm. However, other methods, including physically constraining the location of a microparticles and a cell in a micro-well, are possible. Identification data characterizing the physically associated oligonucleotide and laser particle are stored in a non-volatile storage arrangement. This information together with the corresponding microparticle constitutes a cellular coding construct.

After physical association is established,there is obtained a first measurement of a set of biological data of the cells. During this measurement, the identification data characterizing the oligonucleotide and the laser particle that are physical associated with each cell is also either measured or retrieved. The identification data and biological data characterizing the cellular entity are encoded in the same or another non-volatile storage arrangement. After the first measurement, cells are pooled together or mixed physically. Then, a second measurement is performed on the cells to acquire another set of biological data from the cells. This second set of biological data is also encoded in the non-volatile storage arrangement along with the first set of biological data. Finally, both sets of biological data are analyzed to understand the characteristics of each single cell associated with the same cellular coding construct.

This general workflow provides compelling advantages over the prior art for maximizing the number of parameters that can be measured at one time. This new capability allows most optimized methodologies to be used. Currently, fluorescence microscopy is most suited to obtain spatial information of cells both in isolation and in tissues. Flow cytometry and droplet-based single-cell sequencing offer the highest throughput for proteomic and transcriptomic analysis respectively. LP barcoding is compatible with all of these gold-standard technologies. Cellular information at different levels or dimensions can be obtained using the best measurement modalities, and the datasets are combined to each single cell using the optical barcodes. This approach ensures high throughput, low cost, and high data quality.

FIG. 10B depicts a more specific workflow chart combining optical and sequencing analysis. In this case, cells are tagged with multi-barcoding microparticles, optical measurements of the cells are performed, the entire or subgroup of the measured cells are collected, sequencing of the collected cells is performed, and computational analysis is performed to combine the optical and sequencing data for individual cells. Examples of the optical measurement include imaging and flow cytometry. The optical measurement involves reading the optical barcodes of the cells.

FIG. 10C depicts another workflow chart expanded from the previous example. Here, cells are tagged with multi-barcoding microparticles, a first optical measurement is performed on the cells, which include optical barcode reading, the cells are pooled, a second measurement is performed, which includes optical barcode reading. Single-cell sequencing is performed on the cells. And, then computation analysis combines the first and second optical measurement data and the sequencing data.

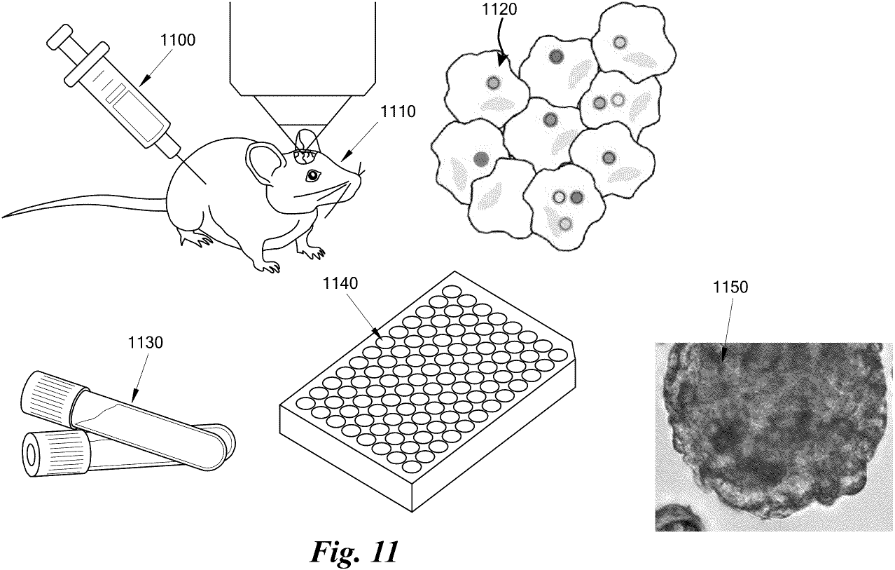

FIG. 11 illustrates different types of cells that can be tagged by barcoded microparticles. LPs are suitable for tagging cells 1100 prior to injection into in vivo systems such as animals 1110, cells in situ in tissues 1120, blood cells 1130 extracted from patients, and cells in 2D and 3D cultures, 1140 and 1150.

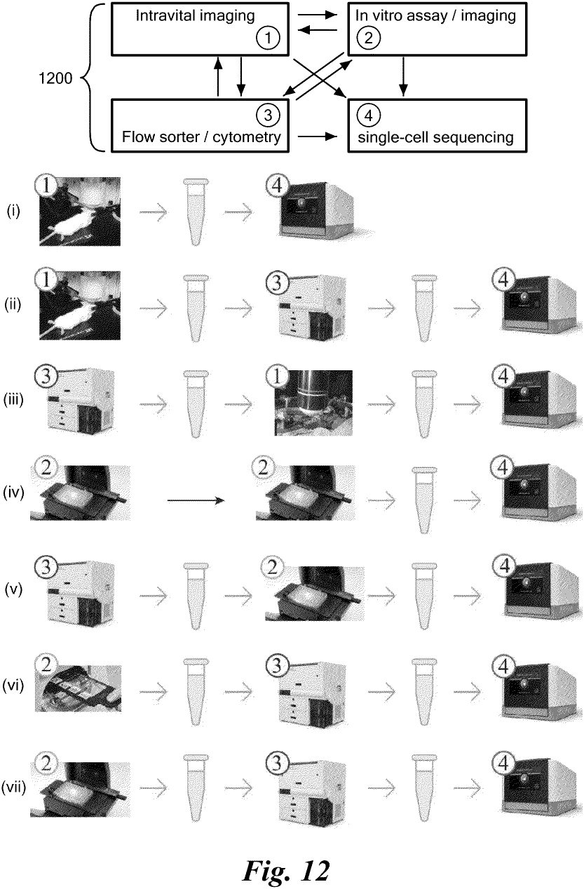

FIG. 12 illustrates more specific examples of the various workflows enabled by the multi-barcoding microparticles. A diagram 1200 illustrates a cross-platform, multidimensional single-cell analysis across in vivo imaging, in vitro assays, flow cytometry, and sequencing. Cells can be analyzed in any orders except for sequencing that is done at the terminal stage. Seven examples, denoted (i) to (vii), are illustrated. A brief description of each example is given below.

Connecting live imaging to molecular omics of individual cells (FIG. 12-i). Observing cells in their native environment in vivo using optical microscopy led to numerous findings that would be difficult to appreciate otherwise. A variety of dynamic processes, such as migration, cell-cell interactions, and cell-tissue interactions, are visualized in real time. Traditionally, genetically encoded fluorescent reporters were used to measure expression of one or a few genes of interest. For further molecular analysis, cells are marked using photoconversion of fluorophores or light-induced printing of DNA barcodes (Zip-Seq). Alternatively, laser capture microdissection can isolate cells from tissue under a microscope and enable subsequent genomic, transcriptomic and proteomic profiling. However, both methods are slow and usable for a limited number of cells. LP barcoding will enable scientists to record the behaviors of a large number of cells and conduct state-of-the-art single-cell sequencing in a high-throughput manner.

CRISPR-based pooled libraries of genetically altered cells is a scalable and programmable technique to explore the connection between gene activity and functional phenotypes of mammalian systems. Current large-scale optical screen methods are limited to in situ sequencing, which is labor intensive, time-consuming, and not widely available at most single-cell sequencing labs. Our strategy tracks live-cell phenotypes, dissociate the cells, and analyze the genetic perturbation using commercially available droplet-based NGS single-cell sequencing platforms (feature barcoding technology of 10x genomics for CRISPR perturbations). Laser particle-based single-cell sequencing can therefore be used for high-throughput, large-scale and dynamic optical pooled genetic perturbation screens.

Connecting in vivo imaging, flow cytometry, and sequencing (FIG. 12-ii). Cells are harvested after in vivo imaging and then analyzed in flow cytometry. This process connects in vivo functional data and high-throughput biomarker analysis. Furthermore, the analyzed cells in flow can be collected and even sorted for further omics analysis. This workflow integrates the three gold standard techniques (microscopy, flow cytometry, and sequencing), and the acquired data are combined for individual cells according to their optical barcode.

Preclinical studies of adoptive cell transfer and cell therapy at single-cell resolution (FIG. 12-iii). Adoptive cell transfer is widely used in studies of immune systems and developments of immunotherapies for diseases such as cancer. In addition, stem-cell therapy hold promise in regenerative medicine. Optical barcoding will enable scientists to observe the behaviors and fate of individual transferred cells in animal disease models. The transferred cells are measured over time before and after therapy with single-cell resolution. This new capability is expected to accelerate the discovery and development of more effective treatments.

In vitro assay and sequencing at the single-cell level (FIG. 12-iv). Cell-based assays are widely used in drug development, helping to bring drugs to the market in a quick and efficient manner. Cell-based assays quantify biological activity, biochemical mechanisms and off-target interactions, as well as cytotoxicity. Optical barcoding enables scientists to perform cell-based assays in vitro using optical microscopy at single-cell resolution and then obtain their molecular omics information, providing unprecedentedly comprehensive information of individual cells. This new workflow could accelerate drug discovery.

High-content drug screening and cell-based assay at the single-cell level (FIG. 12-v). Billions of dollars are invested globally in the clinical approval of new drug compounds, but only a small handful of new chemical entities are approved each year. Current cell-based assays measure cells responses to different compounds in different conditions. A one-time measurement assay, however, often cannot reveal the complex effects of drugs on heterogeneous cell population. Optical barcoding allows measurements at multiple time points tracking the dynamic responses of individual cells. This new capability can be useful in high-content drug screening.

Deep-profiling spatial transcriptomics (FIG. 12-vi). Determining the molecular profiles of single cells in the spatial context of tissues is an important undertaking. Multiplexed FISH techniques have been improved to detect over 1,000 genes in a cell, but at limited throughput. Spatial transcriptomics techniques, such as the recently commercialized Visium platform from 10× Genomics, analyze RNAs collected from tissues using oligo-barcoded slides using the high-throughput deep-profiling single-cell techniques. However, most of the conventional techniques are limited to 2D tissues and do not have true single-cell resolution, as RNAs are captured by 2D patterns with a discrete interval in contact with a tissue. Furthermore, these techniques typically only measure up to tens of genes per cell, compared to thousands of genes per cell in conventional scRNA-seq. LP barcoding of cells in tissues, along with co-labeling with oligo-barcodes, can overcome these limitations.

Deep-profiling spatial proteomics (FIG. 12-vii). Combining spatial transcriptome with protein expression in the same tissue section provides a deeper, more holistic understanding of tissue organization. Protein detection in tissues has traditionally been conducted by fluorescence microscopy using antibody-fluorophore conjugates after tissue fixation and permeabilization. Repeated antibody elution and staining steps or use of oligo-barcoded antibodies extended multiplexed detection. Using barcoded antibodies (DNA-Ab, feature barcodes, or Ab-seq), it is possible to detect more cell-surface proteins (only limited by the availability of antibodies). LP barcoding in conjunction with oligo-barcodes and DNA-Ab can enable the deep profiling of epitopes while providing 3D organization of single cells in tissues.

In addition, there are numerous other combinations of measurements. For examples, flow cytometry can be performed on a sample multiple time with time delays. This workflow is useful to analyze changes in cells over time, after activation, or in response to drugs. U.S. Pat. Application No. 17/166,524 describes cyclic flow cytometry, in which flow cytometry measurement is performed on cells tagged with optical barcoding LPs and changing fluorophore-antibody markers on cells on each flow cytometry cycle. Multi-barcoding microparticles can be used for cyclic flow cytometry and in conjunction with cyclic flow cytometry.

Once the biological data are obtained through the various analyses and aligned to individual cells based on the identification data of cellular constructs, the aligned biological data may also be stored in a non-volatile storage arrangement.

One embodiment of this invention is a non-volatile storage arrangement encoded with data characterizing a set of cellular entities, wherein for each cellular entity there is provided an identifier characterizing a structurally coded oligonucleotide and a laser particle physically associated with the structurally coded oligonucleotide, and for each identifier is provided, pertinent to the corresponding cellular entity, information selected from the group consisting of DNA data, RNA data, protein data, morphology data, location data, functional data, and behavioral data.

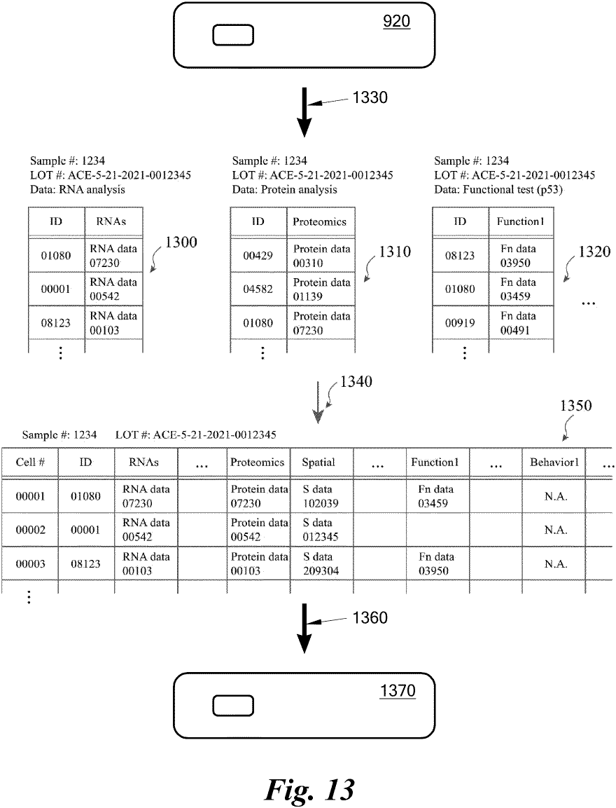

FIG. 13 illustrates this embodiment. As an exemplary illustration, consider an RNA analysis data of a sample no. 1234 in which cells are tagged with dual-barcoding particles from a lot number: ACE-5-21-2021-0012345. The oligo barcodes identified during the RNA analysis allow the RNA data of single cells to be aligned to the identifiers (ID’s) of cellular barcoding constructs associated with the corresponding single cells. Such RNA data 1300 is obtained. Protein data 1310 and functional data (related to p53 activities as an example) 1320 are obtained from flow cytometry and imaging analysis, during which the optical barcodes of the cellular barcoding constructs associated with the corresponding cells were used to determine the associated identifiers of the barcoding constructs. For this process, the identification data encoded in the non-volatile storage medium 920 has been retried 1330 and used.

As the biological data in different dimensions (i.e., RNA, protein, and function) are obtained, the data can be aligned with respect to the identifiers to single cells. This data integration process 1340 produces an integrated dataset 1350 that contain comprehensive biological data of single cells in a large scale. This data is then stored 1360 into a non-volatile storage arrangement 1370, which is then encoded with data characterizing a set of cellular entities.

In this example, it has been implicitly assumed that a single cell is associated with only one identifier and vice versa. However, when a cell is tagged with more than one microparticle, it may be possible that a single cell is associated with more than one identifier. Conversely, it may be possible that a specific identifier is assigned to more than one cell, when two microparticles used for a sample of cells have an identical optical or oligo barcode.

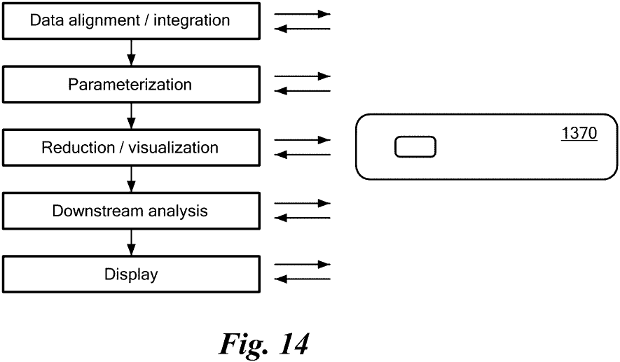

FIG. 14 illustrates various data analysis steps using the integrated data in the non-volatile storage medium 1370. The data alignment and integration are followed by various processes, such as parametrization, data reduction, visualization, downstream analysis, and display. An example of parametrization is to determine parameters or metrics drawn from the integrated biological data. For example, the RNA and protein expression data may be converted to a numerical model with coefficients as new parameters. Data reduction and visualization include principal component analysis (PCA), non-negative matrix factorization, linear discriminant analysis, generalized discriminant analysis, autoencoder, t-distributed stochastic neighbor embedding (t-SNE) analysis, uniform manifold approximation and projection (UMAP) correlation analysis. The downstream analysis may compute correlation, clustering (i.e., heatmap) and feature selection. Finally, these data are displayed on a computer monitor or into electronic files.

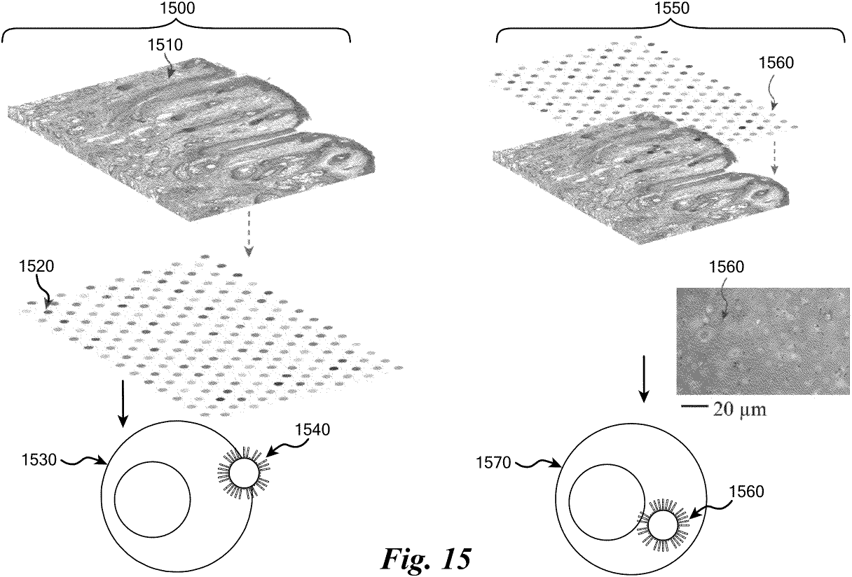

FIG. 15 illustrates two exemplary methods to tag cells in tissues with microparticles. One method 1500 uses a tissue slice sample 1510 and an array of multi-barcoding microparticles 1520. The array may be a two-dimensional periodic or random arrangement of LPs printed on a flat slide or placed on a micro-patterned substrate. Then the tissue and array are brought to physical contact. The surface of each microparticle is configured to stick to the cellular membrane. Some coating methods for exterior cell membrane tagging are described with reference to FIG. 4C. If the tissue is fresh and cells are alive, the microparticles may be internalized into the cells with incubation. Once the tagging is established, the cells are dissociated from the tissue using methods such as trypsinization. Individual cells 1530 tagged with microparticles 1540 are collected for single-cell sequencing.

Alternatively, the barcoding microparticles may be sprayed or dropped onto the tissue surface for tagging. This method 1550 may use a spray nozzle to spread LPs on a fresh tissue surface, which induces cell tagging. This method is suited for 2D mapping of tissue. For 3D mapping, the method 1550 may use a “biolistic” delivery device. Gene guns have been used to deliver DNA coated on 1-2 µm-sized gold microparticles onto plant tissues. As a preliminary demonstration, we have used a gene gun (PDS-1000, Bio-Rad Laboratories) to shoot a large number of microdisk LPs onto a fresh murine tissue. It was found that LPs 1560 can penetrate into the soft tissue at different depths up to 100 µm depending on the air pressure of the gene gun. To minimize RNA degradation, tissues may be maintained at 4° C., and an RNA stabilizer may be used. The tissue is dissociated, and single cells 1570 containing at least one LP is harvested using a flow sorter for single-cell sequencing.

For physical manipulation of barcoding microparticles, the microparticles may further employ magnetic materials, such as iron, nickel, and cobalt. For example, iron nanoparticles with a size of 10-50 nm are coated onto the surface of LPs. Such magnetic microparticles can then be moved, pulled, or pushed using magnets. This ability may be used to facilitate the tagging of microparticles to cells in tissues. Also, magnetic microparticles can help removing untagged or free LPs from samples.

Besides cells as samples, multi-barcoding microparticles can be used to tag subcellular entities, such as nuclei, as illustrated in FIG. 4D. Sequencing mRNA in cell nuclei is currently applied for various applications including epigenetic analysis and measuring RNA velocity. Single cell ATAC-seq is currently accomplished by isolating single cell nuclei and performing tagmentation using a Tn5 transposase to insert sequencing adapters into open regions of chromatin. Each nucleus is encapsulated with a barcoded bead, similar to 100 in FIG. 5A, which contains oligonucleotide barcoding strands capable of capturing the tagmented DNA. Nuclear tagging with multi-barcoding LPs could enable novel multidimensional ATAC-seq workflows.

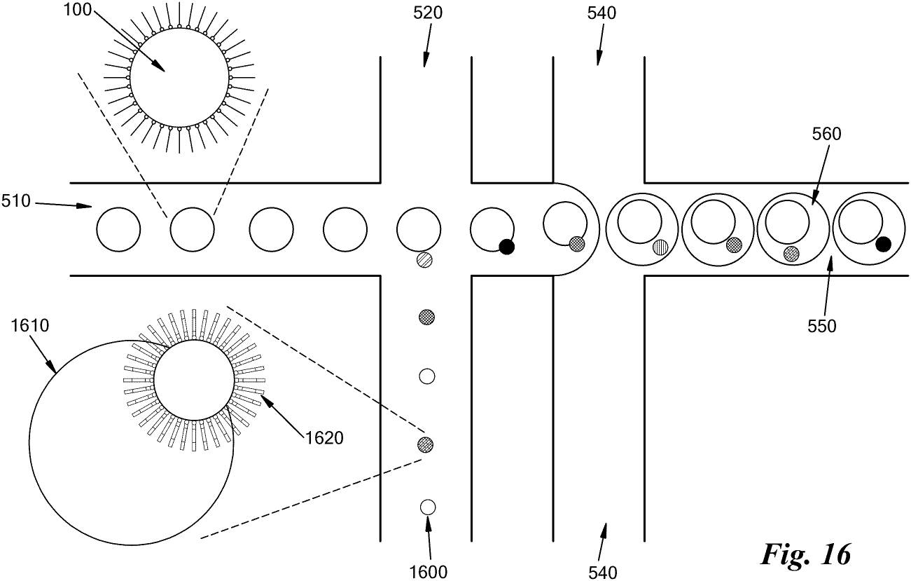

FIG. 16 illustrates an exemplary embodiment for nuclei sequencing, which is nearly identical to the embodiment described in connection with FIG. 5A, but differs in that the sample 1600 is an individual cellular nucleus 1610 tagged with a multi-barcoding microparticle 1620.

In addition to the droplet-based single-cell sequencing techniques, the barcoding microparticles are compatible with various other techniques. Examples of the non-droplet-based techniques include those based on separating single cells into wells on a plate, such as SMART-Seq, SMART-Seq2, and Seq-Well.

The embodiments described so far benefit from making all the optical and oligo barcodes of the microparticles to be different from each other, so that individual cells and cellular entities are distinguished from each other. Instead of this unique-barcoding scheme, a group-barcoding or sample-barcoding scheme may be useful for certain applications, where a group of barcoding particles share a common optical or oligonucleotide feature that is uniquely assigned to the specific group. One analogy method is cell hashing that uses a series of oligo-tagged antibodies against ubiquitously expressed surface proteins with different barcodes to uniquely label cells from distinct samples. These samples be subsequently pooled in one single-cell sequencing. Cell hashing is used for sample multiplexing and super-loading. Another analogy is labeling different cell groups with fluorescent proteins with distinct colors. This multi-color technique is used for visualizing the location and dynamics of the cells using fluorescent microscopy, for example.

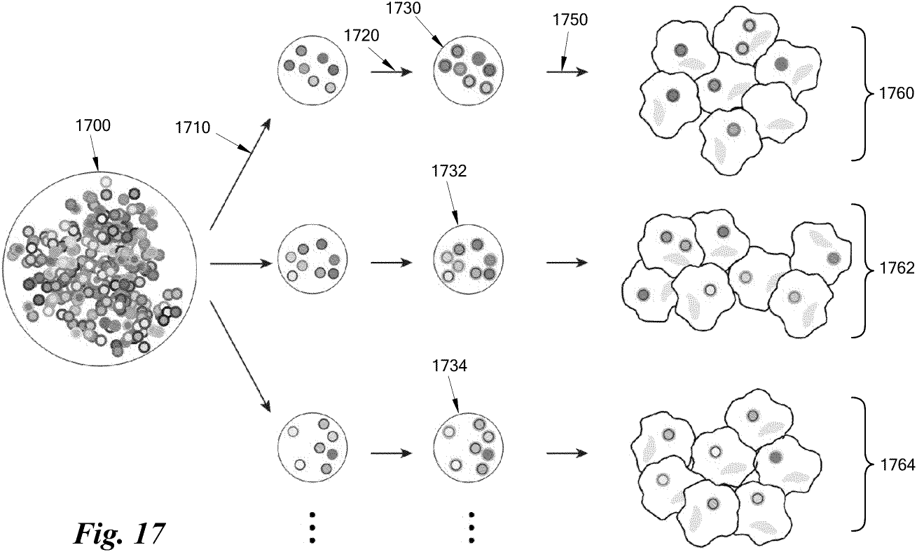

FIG. 17 depicts an embodiment to produce such barcoding microparticles suitable for sample barcoding or sample multiplexing. A large number of barcoding microparticles 1700 with optical and oligo barcodes are prepared. In process 1710, these microparticles are split into different wells or containers. In process 1720, microparticles in different wells are then coupled, linked, attached, or coated with group-specific oligo sequences 1720. The group oligo barcodes in distinct wells 1730, 1732, and 1734 are mutually distinct. All the microparticles in the same well share an identical group oligo barcode. The microparticles arranged or stored in groups can then be used for tagging, 1750, multiple samples 1760, 1762, and 1764. The group barcodes facilitate identifying and distinguishing the groups of samples. Although the unique-barcoding scheme can in principle be used to label multiple samples or groups, this group-barcoding scheme can reduce the errors in identifying different groups and even different cells within a group.

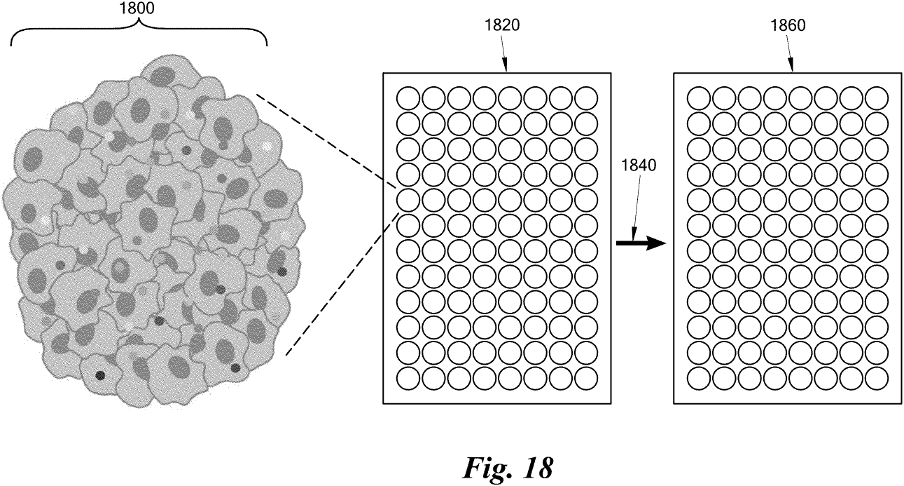

The multi-barcoding microparticles facilitate the task of matching the optical barcodes and oligo barcodes. However, the multi-barcoding strategy can be achieved without attaching oligonucleotide barcodes directly on the surface of optical barcoding microparticles. FIG. 18 depicts one such a method based on a split-pool cellular barcoding technique, known as single-cell combinatorial indexing RNA sequencing or sci-SEQ, or split-pool ligation-based transcriptome sequencing (SPLiT-seq). Sci-SEQ is a combinatorial indexing strategy relying on split-pool barcoding. In each stage of splitting, first and second oligo barcode sequences are added and ligated in a manner similar to that described in connection with FIG. 3B. Briefly, cells in a sample are tagged with optical microparticles. The tagged cells 1800 are then combinatorically indexed into individual wells of a 96-well or 384-well plate 1820. A microfluidic system deposits each cell while simultaneously reading out its optical barcode in a manner analogous to fluorescence-based indexing techniques currently performed by traditional flow-cytometry devices. Nucleotide-based barcode tags, such as barcoded polythymidine primers, is introduced to the individual groups of cells populating each well. Subsequent pooling 1840 and re-splitting into a well plate 1860 establish an association between the transcriptomic profile eventually determined by sequencing and the original location of the optically barcoded cell.

The embodiments of the invention described above are intended to be merely exemplary; numerous variations and modifications will be apparent to those skilled in the art. All such variations and modifications are intended to be within the scope of the present invention as defined in any appended claims.

The following references constitute a part of the present application.

1. Martino, N., et al. Wavelength-encoded laser particles for massively multiplexed cell tagging. Nature Photonics 13, 720-+ (2019).

2. Kwok, S.J.J., Martino, N., Dannenberg, P.H. & Yun, S.H. Multiplexed laser particles for spatially resolved single-cell analysis. Light-Science & Applications 8(2019).

3. Macosko, E.Z., et al. Highly Parallel Genome-wide Expression Profiling of Individual Cells Using Nanoliter Droplets. Cell 161, 1202-1214 (2015).

4. Klein, A.M., et al. Droplet barcoding for single-cell transcriptomics applied to embryonic stem cells. Cell 161, 1187-1201 (2015).

5. Gierahn, T.M., et al. Seq-Well: portable, low-cost RNA sequencing of single cells at high throughput. Nat Methods 14, 395-398 (2017).

6. Stoeckius, M., et al. Simultaneous epitope and transcriptome measurement in single cells. Nat Methods 14, 865-868 (2017).

7. Peterson, V.M., et al. Multiplexed quantification of proteins and transcripts in single cells. Nat Biotechnol 35, 936-939 (2017).

8. Levy, L., Sahoo, Y., Kim, K.-S., Bergey, E.J. & Prasad, P.N. Nanochemistry: Synthesis and Characterization of Multifunctional Nanoclinics for Biological Applications. Chemistry of Materials 14, 3715-3721 (2002).

9. Nguyen, C.V., et al. Preparation of Nucleic Acid Functionalized Carbon Nanotube Arrays. Nano Letters 2, 1079-1081 (2002).

10. Mangalam, A.P., Simonsen, J. & Benight, A.S. Cellulose/DNA Hybrid Nanomaterials. Biomacromolecules 10, 497-504 (2009).

11. Zilionis, R., et al. Single-cell barcoding and sequencing using droplet microfluidics. Nat Protoc 12, 44-73 (2017).

12. Xia, T.A., et al. Polyethyleneimine Coating Enhances the Cellular Uptake of Mesoporous Silica Nanoparticles and Allows Safe Delivery of siRNA and DNA Constructs. ACS Nano 3, 3273-3286 (2009).

13. Kimmerling, R.J., et al. Linking single-cell measurements of mass, growth rate, and gene expression. Genome Biol 19, 207 (2018).

14. Buenrostro, J.D., et al. Single-cell chromatin accessibility reveals principles of regulatory variation. Nature 523, 486-490 (2015).

15. Hu, Fanghao, et al. Supermultiplexed optical imaging and barcoding with engineered polyynes. Nat Methods 15, 194-200 (2018).

16. Huy Q., et al. Nguyen, Programmable Microfluidic Synthesis of Over One Thousand Uniquely Identifiable Spectral Codes. Adv. Opt. Materials 5, 1600548 (2017).

What is claimed is:

1. A cellular coding construct that uniquely codes a cellular entity, the cellular coding construct comprising:
a laser particle; and
a structurally coded oligonucleotide,
wherein the structurally coded oligonucleotide and the laser particle have a physical association with each other and are configured for physical association with the cellular entity and also configured for distinctive identification of the cellular entity.

2. A cellular construct according to claim 1, further comprising a non-volatile storage arrangement encoded with identification data characterizing the structurally coded oligonucleotide and the laser particle and their physical association.

3. A cellular construct according to claim 1, wherein the laser particle and the structurally coded oligonucleotide have a combined dimension that is less than 3 µm.

4. A cellular construct according to claim 1, wherein the cellular construct is physically associated with a specified cellular entity.

5. A cellular construct according to claim 2, wherein the cellular construct is physically associated with a specified cellular entity and wherein the non-volatile storage arrangement is further encoded with biological data characterizing the specified cellular entity.

6. A cellular construct according to claim 5, wherein the biological data is genetic sequence data.

7. A cellular construct according to claim 4, wherein the cellular construct is configured for machine readout of identification data.

8. A cellular construct according to claim 4, wherein the combined cellular construct and specified cellular entity are configured for machine readout of data relating to the specified cellular entity.

9. A cellular construct according to claim 8, wherein the cellular construct is physically associated with a specified cellular entity and wherein the non-volatile storage arrangement is further encoded with biological data characterizing the specified cellular entity.

10. A cellular construct according to claim 1, wherein the cellular construct further includes a linker configured to physically attach the cellular construct to the cellular entity.

11. A population of objects, wherein each object is a distinct cellular construct according to claim 1.

12. A cellular construct according to claim 1, wherein the structurally coded oligonucleotide includes a plurality of ligated sequence segments.

13. A cellular construct according to claim 1, wherein the physical association between the structurally coded oligonucleotide and the laser particle is configured for physical disassociation.

14. A non-volatile storage arrangement encoded with data characterizing a set of cellular entities, wherein for each cellular entity there is provided an identifier characterizing a structurally coded oligonucleotide and a laser particle physically associated with the structurally coded oligonucleotide, and for each identifier is provided, pertinent to the corresponding cellular entity, information selected from the group consisting of DNA data, RNA data, protein data, morphology data, location data, functional data, and behavioral data.

15. A non-volatile storage arrangement according to claim 14, wherein the structurally coded oligonucleotide and the laser particle are physically associated with the cellular entity.


SEQUENCE LISTING (36 sequences)

SEQ ID NO: 1 - DNA, 89 bp, synthetic construct
  source 1..89: mol_type = other DNA
  modified_base 1: mod_base = OTHER; note = 5' Amino Modifier C12 nucleotide
  misc_difference 35: note = a, c, g, or t
  misc_difference 36: note = a, c, g, or t
  misc_difference 37: note = a, c, g, or t
  misc_difference 38: note = a, c, g, or t
  misc_difference 39: note = a, c, g, or t
  misc_difference 40: note = a, c, g, or t
  misc_difference 41: note = a, c, g, or t
  misc_difference 42: note = a, c, g, or t
  misc_difference 43: note = a, c, g, or t
  misc_difference 44: note = a, c, g, or t
  misc_difference 45: note = a, c, g, or t
  misc_difference 46: note = a, c, g, or t
  misc_difference 47: note = a, c, g, or t
  misc_difference 48: note = a, c, g, or t
  misc_difference 49: note = a, c, g, or t
  misc_difference 50: note = a, c, g, or t
  misc_difference 51: note = a, c, g, or t
  misc_difference 52: note = a, c, g, or t
  misc_difference 53: note = a, c, g, or t
  misc_difference 54: note = a, c, g, or t
  misc_difference 55: note = a, c, g, or t
  misc_difference 56: note = a, c, g, or t
  misc_difference 57: note = a, c, g, or t
  misc_difference 58: note = a, c, g, or t
  misc_difference 59: note = a, c, g, or t
  misc_difference 60: note = a, c, g, or t
  misc_difference 61: note = a, c, g, or t
  misc_difference 62: note = a, c, g, or t
  misc_difference 63: note = a, c, g, or t
  misc_difference 64: note = a, c, g, or t
  misc_difference 65: note = a, c, g, or t
  misc_difference 66: note = a, c, g, or t
  misc_difference 67: note = a, c, g, or t
  modified_base 87: mod_base = OTHER; note = nucleotide with phosphorothioate linkage
  modified_base 88: mod_base = OTHER; note = nucleotide with phosphorothioate linkage
gtgactggagttcagacgtgtgctcttccgatctnnnnnnnnnnnnnnnnnnnnnnnnnnnnnnnnngctttaaggccggtcctagcaa

SEQ ID NO: 2 - DNA, 92 bp, synthetic construct
  source 1..92: mol_type = other DNA
  modified_base 1: mod_base = OTHER; note = 5' Amino Modifier C12 nucleotide
  modified_base 90: mod_base = OTHER; note = nucleotide with phosphorothioate linkage
  modified_base 91: mod_base = OTHER; note = nucleotide with phosphorothioate linkage
gctagttcccttggcacccgagaattcccactgaactcatcgcattcgctcacgtcgatbaaaaaaaaaaaaaaaaaaaaaaaaaaaaaaaa

SEQ ID NO: 3 - DNA, 80 bp, synthetic construct
  source 1..80: mol_type = other DNA
ctacacgacgctcttccgatctaaacctgagaaaccgcctgttcgtatcgtttttttttttttttttttttttttttttt

SEQ ID NO: 4 - DNA, 20 bp, synthetic construct
  source 1..20: mol_type = other DNA
ccttggcacccgagaattcc

SEQ ID NO: 5 - DNA, 134 bp, synthetic construct
  source 1..134: mol_type = other DNA
ccttggcacccgagaattcccactgaactcatcgcattcgctcacgtcgatbaaaaaaaaaaaaaaaaaaaaaaaaaaaaaaaacgatacgaacaggcggtttctcaggtttagatcggaagagcgtcgtgtag

SEQ ID NO: 6 - DNA, 20 bp, synthetic construct
  source 1..20: mol_type = other DNA
  modified_base 1: mod_base = OTHER; note = 5' 6-fluorescein amidite modified nucleotide
vttttttttttttttttttt

SEQ ID NO: 7 - DNA, 44 bp, synthetic construct
  source 1..44: mol_type = other DNA
  modified_base 1: mod_base = OTHER; note = Amide modified nucleotide
gtgactggagttcagacgtgtgctcttccgatctaagattgcac

SEQ ID NO: 8 - DNA, 26 bp, synthetic construct
  source 1..26: mol_type = other DNA
gtgactggagttcagacgtgtgctct

SEQ ID NO: 9 - DNA, 18 bp, synthetic construct
  source 1..18: mol_type = other DNA
tccgatctaagattgcac

SEQ ID NO: 10 - DNA, 49 bp, synthetic construct
  source 1..49: mol_type = other DNA
  misc_difference 17: note = a, c, g, or t
  misc_difference 18: note = a, c, g, or t
  misc_difference 19: note = a, c, g, or t
  misc_difference 20: note = a, c, g, or t
  misc_difference 21: note = a, c, g, or t
  misc_difference 22: note = a, c, g, or t
  misc_difference 23: note = a, c, g, or t
  misc_difference 24: note = a, c, g, or t
  misc_difference 25: note = a, c, g, or t
  misc_difference 26: note = a, c, g, or t
  misc_difference 27: note = a, c, g, or t
  misc_difference 28: note = a, c, g, or t
  misc_difference 29: note = a, c, g, or t
  misc_difference 30: note = a, c, g, or t
  misc_difference 31: note = a, c, g, or t
caacatcagatgctcannnnnnnnnnnnnnngtgcaatcttagatcgga

SEQ ID NO: 11 - DNA, 52 bp, synthetic construct
  source 1..52: mol_type = other DNA
  misc_difference 23: note = a, c, g, or t
  misc_difference 24: note = a, c, g, or t
  misc_difference 25: note = a, c, g, or t
  misc_difference 26: note = a, c, g, or t
  misc_difference 27: note = a, c, g, or t
  misc_difference 28: note = a, c, g, or t
  misc_difference 29: note = a, c, g, or t
  misc_difference 30: note = a, c, g, or t
  misc_difference 31: note = a, c, g, or t
  misc_difference 32: note = a, c, g, or t
  misc_difference 33: note = a, c, g, or t
  misc_difference 34: note = a, c, g, or t
  misc_difference 35: note = a, c, g, or t
  misc_difference 36: note = a, c, g, or t
ttgctaggaccggccttaaagcnnnnnnnnnnnnnncaacatcagatgctca

SEQ ID NO: 12 - DNA, 109 bp, synthetic construct
  source 1..109: mol_type = other DNA
  modified_base 1: mod_base = OTHER; note = Amide modified nucleotide
  misc_difference 45: note = a, c, g, or t
  misc_difference 46: note = a, c, g, or t
  misc_difference 47: note = a, c, g, or t
  misc_difference 48: note = a, c, g, or t
  misc_difference 49: note = a, c, g, or t
  misc_difference 50: note = a, c, g, or t
  misc_difference 51: note = a, c, g, or t
  misc_difference 52: note = a, c, g, or t
  misc_difference 53: note = a, c, g, or t
  misc_difference 54: note = a, c, g, or t
  misc_difference 55: note = a, c, g, or t
  misc_difference 56: note = a, c, g, or t
  misc_difference 57: note = a, c, g, or t
  misc_difference 58: note = a, c, g, or t
  misc_difference 74: note = a, c, g, or t
  misc_difference 75: note = a, c, g, or t
  misc_difference 76: note = a, c, g, or t
  misc_difference 77: note = a, c, g, or t
  misc_difference 78: note = a, c, g, or t
  misc_difference 79: note = a, c, g, or t
  misc_difference 80: note = a, c, g, or t
  misc_difference 81: note = a, c, g, or t
  misc_difference 82: note = a, c, g, or t
  misc_difference 83: note = a, c, g, or t
  misc_difference 84: note = a, c, g, or t
  misc_difference 85: note = a, c, g, or t
  misc_difference 86: note = a, c, g, or t
  misc_difference 87: note = a, c, g, or t
gtgactggagttcagacgtgtgctcttccgatctaagattgcacnnnnnnnnnnnnnngagcatctgatgttgnnnnnnnnnnnnnngctttaaggccggtcctagcaa

SEQ ID NO: 13 - DNA, 72 bp, synthetic construct
  source 1..72: mol_type = other DNA
gtcagatgtgtataagagacagaaacctgagaaaccgcctgttcgtatcgttgctaggaccggccttaaagc

SEQ ID NO: 14 - DNA, 34 bp, synthetic construct
  source 1..34: mol_type = other DNA
gtgactggagttcagacgtgtgctcttccgatct

SEQ ID NO: 15 - DNA, 22 bp, synthetic construct
  source 1..22: mol_type = other DNA
gtcagatgtgtataagagacag

SEQ ID NO: 16 - DNA, 161 bp, synthetic construct
  source 1..161: mol_type = other DNA
  misc_difference 45: note = a, c, g, or t
  misc_difference 46: note = a, c, g, or t
  misc_difference 47: note = a, c, g, or t
  misc_difference 48: note = a, c, g, or t
  misc_difference 49: note = a, c, g, or t
  misc_difference 50: note = a, c, g, or t
  misc_difference 51: note = a, c, g, or t
  misc_difference 52: note = a, c, g, or t
  misc_difference 53: note = a, c, g, or t
  misc_difference 54: note = a, c, g, or t
  misc_difference 55: note = a, c, g, or t
  misc_difference 56: note = a, c, g, or t
  misc_difference 57: note = a, c, g, or t
  misc_difference 58: note = a, c, g, or t
  misc_difference 59: note = a, c, g, or t
  misc_difference 76: note = a, c, g, or t
  misc_difference 77: note = a, c, g, or t
  misc_difference 78: note = a, c, g, or t
  misc_difference 79: note = a, c, g, or t
  misc_difference 80: note = a, c, g, or t
  misc_difference 81: note = a, c, g, or t
  misc_difference 82: note = a, c, g, or t
  misc_difference 83: note = a, c, g, or t
  misc_difference 84: note = a, c, g, or t
  misc_difference 85: note = a, c, g, or t
  misc_difference 86: note = a, c, g, or t
  misc_difference 87: note = a, c, g, or t
  misc_difference 88: note = a, c, g, or t
  misc_difference 89: note = a, c, g, or t
gtgactggagttcagacgtgtgctcttccgatctaagattgcacnnnnnnnnnnnnnnntgagcatctgatgttgnnnnnnnnnnnnnngctttaaggccggtcctagcaacgatacgaacaggcggtttctcaggtttctgtctcttatacacatctgac

SEQ ID NO: 17 - DNA, 34 bp, synthetic construct; identical to SEQ ID NO: 14
  source 1..34: mol_type = other DNA
  modified_base 1: mod_base = OTHER; note = 5' Amino Modifier C12 nucleotide

SEQ ID NO: 18 - DNA, 21 bp, synthetic construct
  source 1..21: mol_type = other DNA
  modified_base 1: mod_base = OTHER; note = 5'-phosphate modified nucleotide
  misc_difference 7: note = a, c, g, or t
  misc_difference 8: note = a, c, g, or t
  misc_difference 9: note = a, c, g, or t
  misc_difference 10: note = a, c, g, or t
  misc_difference 11: note = a, c, g, or t
  misc_difference 12: note = a, c, g, or t
  misc_difference 13: note = a, c, g, or t
  misc_difference 14: note = a, c, g, or t
acatggnnnnnnnntatctac

SEQ ID NO: 19 - DNA, 20 bp, synthetic construct
  source 1..20: mol_type = other DNA
ccatgtagatcggaagagca

SEQ ID NO: 20 - DNA, 35 bp, synthetic construct
  source 1..35: mol_type = other DNA
  modified_base 1: mod_base = OTHER; note = 5'-phosphate modified nucleotide
  misc_difference 7: note = a, c, g, or t
  misc_difference 8: note = a, c, g, or t
  misc_difference 9: note = a, c, g, or t
  misc_difference 10: note = a, c, g, or t
  misc_difference 11: note = a, c, g, or t
  misc_difference 12: note = a, c, g, or t
  misc_difference 13: note = a, c, g, or t
  modified_base 33: mod_base = OTHER; note = nucleotide with phosphorothioate linkage
  modified_base 34: mod_base = OTHER; note = nucleotide with phosphorothioate linkage
gtcacgnnnnnnngctttaaggccggtcctagcaa

SEQ ID NO: 21 - DNA, 13 bp, synthetic construct
  source 1..13: mol_type = other DNA
cgtgacgtagata

SEQ ID NO: 22 - DNA, 90 bp, synthetic construct
  source 1..90: mol_type = other DNA
  modified_base 1: mod_base = OTHER; note = 5' Amino Modifier C12 nucleotide
  misc_difference 41: note = a, c, g, or t
  misc_difference 42: note = a, c, g, or t
  misc_difference 43: note = a, c, g, or t
  misc_difference 44: note = a, c, g, or t
  misc_difference 45: note = a, c, g, or t
  misc_difference 46: note = a, c, g, or t
  misc_difference 47: note = a, c, g, or t
  misc_difference 48: note = a, c, g, or t
  misc_difference 62: note = a, c, g, or t
  misc_difference 63: note = a, c, g, or t
  misc_difference 64: note = a, c, g, or t
  misc_difference 65: note = a, c, g, or t
  misc_difference 66: note = a, c, g, or t
  misc_difference 67: note = a, c, g, or t
  misc_difference 68: note = a, c, g, or t
  modified_base 88: mod_base = OTHER; note = nucleotide with phosphorothioate linkage
  modified_base 89: mod_base = OTHER; note = nucleotide with phosphorothioate linkage
gtgactggagttcagacgtgtgctcttccgatctacatggnnnnnnnntatctacgtcacgnnnnnnngctttaaggccggtcctagcaa

SEQ ID NO: 23 - DNA, 24 bp, synthetic construct
  source 1..24: mol_type = other DNA
gtgactggagttcagacgtgtgct

SEQ ID NO: 24 - DNA, 20 bp, synthetic construct
  source 1..20: mol_type = other DNA
ttgctaggaccggccttaaa

SEQ ID NO: 25 - DNA, 20 bp, synthetic construct
  source 1..20: mol_type = other DNA
  modified_base 1: mod_base = OTHER; note = 5'-phosphate modified nucleotide
  misc_difference 7: note = a, c, g, or t
  misc_difference 8: note = a, c, g, or t
  misc_difference 9: note = a, c, g, or t
  misc_difference 10: note = a, c, g, or t
  misc_difference 11: note = a, c, g, or t
  misc_difference 12: note = a, c, g, or t
  misc_difference 13: note = a, c, g, or t
gtcacgnnnnnnngatgaat

SEQ ID NO: 26 - DNA, 35 bp, synthetic construct
  source 1..35: mol_type = other DNA
  misc_difference 7: note = a, c, g, or t
  misc_difference 8: note = a, c, g, or t
  misc_difference 9: note = a, c, g, or t
  misc_difference 10: note = a, c, g, or t
  misc_difference 11: note = a, c, g, or t
  misc_difference 12: note = a, c, g, or t
  misc_difference 13: note = a, c, g, or t
  modified_base 33: mod_base = OTHER; note = nucleotide with phosphorothioate linkage
  modified_base 34: mod_base = OTHER; note = nucleotide with phosphorothioate linkage
acggcgnnnnnnngctttaaggccggtcctagcaa

SEQ ID NO: 27 - DNA, 13 bp, synthetic construct
  source 1..13: mol_type = other DNA
cgccgtattcatc

SEQ ID NO: 28 - DNA, 109 bp, synthetic construct
  source 1..109: mol_type = other DNA
  misc_difference 41: note = a, c, g, or t
  misc_difference 42: note = a, c, g, or t
  misc_difference 43: note = a, c, g, or t
  misc_difference 44: note = a, c, g, or t
  misc_difference 45: note = a, c, g, or t
  misc_difference 46: note = a, c, g, or t
  misc_difference 47: note = a, c, g, or t
  misc_difference 61: note = a, c, g, or t
  misc_difference 62: note = a, c, g, or t
  misc_difference 63: note = a, c, g, or t
  misc_difference 64: note = a, c, g, or t
  misc_difference 65: note = a, c, g, or t
  misc_difference 66: note = a, c, g, or t
  misc_difference 67: note = a, c, g, or t
  misc_difference 81: note = a, c, g, or t
  misc_difference 82: note = a, c, g, or t
  misc_difference 83: note = a, c, g, or t
  misc_difference 84: note = a, c, g, or t
  misc_difference 85: note = a, c, g, or t
  misc_difference 86: note = a, c, g, or t
  misc_difference 87: note = a, c, g, or t
  modified_base 107: mod_base = OTHER; note = nucleotide with phosphorothioate linkage
  modified_base 108: mod_base = OTHER; note = nucleotide with phosphorothioate linkage
gtgactggagttcagacgtgtgctcttccgatctacatggnnnnnnntatctacgtcacgnnnnnnngatgaatacggcgnnnnnnngctttaaggccggtcctagcaa

SEQ ID NO: 29 - DNA, 159 bp, synthetic construct
  source 1..159: mol_type = other DNA
  misc_difference 41: note = a, c, g, or t
  misc_difference 42: note = a, c, g, or t
  misc_difference 43: note = a, c, g, or t
  misc_difference 44: note = a, c, g, or t
  misc_difference 45: note = a, c, g, or t
  misc_difference 46: note = a, c, g, or t
  misc_difference 47: note = a, c, g, or t
  misc_difference 61: note = a, c, g, or t
  misc_difference 62: note = a, c, g, or t
  misc_difference 63: note = a, c, g, or t
  misc_difference 64: note = a, c, g, or t
  misc_difference 65: note = a, c, g, or t
  misc_difference 66: note = a, c, g, or t
  misc_difference 67: note = a, c, g, or t
  misc_difference 81: note = a, c, g, or t
  misc_difference 82: note = a, c, g, or t
  misc_difference 83: note = a, c, g, or t
  misc_difference 84: note = a, c, g, or t
  misc_difference 85: note = a, c, g, or t
  misc_difference 86: note = a, c, g, or t
  misc_difference 87: note = a, c, g, or t
gtgactggagttcagacgtgtgctcttccgatctacatggnnnnnnntatctacgtcacgnnnnnnngatgaatacggcgnnnnnnngctttaaggccggtcctagcaacgatacgaacaggcggtttctcaggtttctgtctcttatacacatctgac

SEQ ID NO: 30 - DNA, 15 bp, synthetic construct
  source 1..15: mol_type = other DNA
gaggaatatgtaaca

SEQ ID NO: 31 - DNA, 14 bp, synthetic construct
  source 1..14: mol_type = other DNA
ctctggcctggata

SEQ ID NO: 32 - DNA, 15 bp, synthetic construct
  source 1..15: mol_type = other DNA
acacttatattaggc

SEQ ID NO: 33 - DNA, 15 bp, synthetic construct
  source 1..15: mol_type = other DNA
cgagtctaaggcgac

SEQ ID NO: 34 - DNA, 15 bp, synthetic construct
  source 1..15: mol_type = other DNA
cgttgagtacttcca

SEQ ID NO: 35 - DNA, 14 bp, synthetic construct
  source 1..14: mol_type = other DNA
tacagcgcaggtga

SEQ ID NO: 36 - DNA, 14 bp, synthetic construct
  source 1..14: mol_type = other DNA
gttgaagcagattc